(19) United States
(12) Patent Application Publication
(10) Pub. No.: US 2011/0269647 A1
(43) Pub. Date: Nov. 3, 2011

(54) METHOD

(75) Inventors: JERNEJ ULE, Sawston (GB); Julian König, Cambridge (GB)

(73) Assignee: Medical Research Council, Swindon (GB)

(21) Appl. No.: 13/095,643

(22) Filed: Apr. 27, 2011

Related U.S. Application Data
(60) Provisional application No. 61/329,042, filed on Apr. 28, 2010.

(51) Int. Cl. C40B 50/06; C12Q 1/68

(52) U.S. Cl. 506/26; 435/6.19

(57) ABSTRACT

There is described a method for identifying an interaction between an RNA and an RNA binding protein in a biological sample, comprising the steps of: a) contacting the biological sample with an agent that creates a covalent bond between the RNA and the RNA binding protein; b) fragmenting said RNA; c) ligating a first adapter to the fragmented RNA; d) hybridising a reverse transcription primer to said first adapter and reverse transcribing said cross-linked RNA; e) circularising the transcribed cDNA; f) linearising the circularised cDNA; and g) determining the sequence of one or more of the cDNAs.

### FIELD OF INVENTION

The present invention relates to the field of molecular biology. In particular, the present invention relates to the identification of one or more interactions between an RNA and an RNA binding protein in a biological sample.

### BACKGROUND

The interaction of proteins with RNA molecules is of biological and clinical importance, including infections by RNA viruses, translation and mRNA splicing. A certain subgroup of proteins is known to bind RNA molecules, as reviewed by Frankel, et al. Cell 67:1041-1046 (1991). Nucleic acid binding proteins constitute about 23% of the functionally annotated human genes, which reflects the fundamental role that these proteins play in the control of gene expression. To unravel the gene expression networks that they regulate it is necessary to be able to precisely identify these protein-nucleic acid interactions in intact cells.

A major source of proteomic diversity in multicellular eukaryotes is the production of multiple mRNA isoforms. In humans, it was recently estimated that 95-100% of all multi-exon transcripts undergo alternative splicing¹. Splice-site selection is primarily mediated by RNA-binding proteins that bind regulatory elements within nascent transcripts^{2,3}. Heterogeneous nuclear ribonucleoprotein C1/C2 (hnRNP C) was identified over 30 years ago as a core component of hnRNP particles that form on all nascent transcripts⁴. However, although hnRNP C is one of the most abundant proteins in the nucleus, its role in splicing regulation remained unresolved. Whereas some studies suggested that hnRNP particles generally facilitate splicing^{5,6}, individual hnRNP proteins were thought to function as splicing silencers^{7,8}. Resolving these seemingly contradictory observations was hindered by the inability to locate precisely hnRNP particles on nascent transcripts in vivo. In particular, genome-wide mapping of hnRNP C positioning would provide critical information on how hnRNP particles control splicing. Since these highly abundant particles are likely to constitute a general platform for other splicing regulators, deciphering their function would greatly advance our understanding of splicing regulation.

A variety of approaches have been used to study RNA-protein interactions. In vitro approaches have included physical methods—such as x-ray crystallography, and biochemical assays—such as chemical and enzymatic footprinting, gel retardation and filter binding experiments. In vivo approaches for determining RNA-protein interactions are more limited. In vivo cross-linking has been used to assist in the definition of sites of direct contact between nucleic acid and protein. One method that utilises cross linking methodology is called CLIP (UV-crosslinking and immunoprecipitation) and has been used for the isolation of RNA-binding sites of proteins in tissues and cell cultures. CLIP combined with high-throughput sequencing was previously used to generate transcriptome-wide binding maps of several RNA-binding proteins⁹⁻¹². However, since identification of binding sites relied on the analysis of overlapping sequence clusters, distances of less than 30 nucleotides were not resolved. An additional disadvantage of CLIP is the requirement of reverse transcription to pass over residual amino acids that remain covalently attached to the RNA at the cross-link site. Primer extension assays have shown that the vast majority of cDNAs prematurely truncate immediately before the ‘cross-link nucleotide’¹³.

One of the limitations of the CLIP method is that the identification of binding sites relies on analysis of overlapping sequence clusters such that distances of less than 30 nucleotides cannot be resolved. Consequently, it is not possible to precisely identify the point of interaction between an RNA and an RNA binding protein. Another problem with the CLIP method is that reverse transcription is required to pass over residual amino acids that remain covalently attached to the RNA at the crosslink site. Primer extension assays have shown that the vast majority of cDNAs prematurely truncate immediately before the ‘crosslink nucleotide’¹⁰.

### SUMMARY ASPECTS OF THE INVENTION

The present invention describes a method for determining one or more (eg. a plurality of) interactions between an RNA binding protein and RNA, referred to herein as individual-nucleotide resolution CLIP (iCLIP). The method provides for the precise identification and/or mapping of the point of interaction between an RNA binding protein and RNA. Accordingly, the protein-RNA interaction(s) can be identified at, for example, single nucleotide resolution. Aspects and embodiments of the present invention are presented in the accompanying claims.

In a first aspect, there is provided a method for identifying an interaction between an RNA and an RNA binding protein in a biological sample, comprising the steps of: a) contacting the biological sample with an agent that creates a covalent bond between the RNA and the RNA binding protein; b) fragmenting said RNA; c) ligating a first adapter to the fragmented RNA; d) hybridising a reverse transcription primer to said first adapter and reverse transcribing said cross-linked RNA; e) circularising the transcribed cDNA; f) linearising the circularised cDNA; and g) determining the sequence of one or more of the cDNAs.

In a second aspect, there is provided a method for preparing a cDNA library representative of one or more interactions between an RNA and an RNA binding protein, comprising the steps of: a) contacting the biological sample with an agent that creates a covalent bond between the RNA and the RNA binding protein; b) fragmenting said RNA; c) ligating a first adapter to the fragmented RNA; d) hybridising a reverse transcription primer to said first adapter and reverse transcribing said cross-linked RNA; e) circularising the transcribed cDNA; f) optionally linearising the circularised cDNA; and g) optionally sub-cloning the linearised cDNA into a vector.

In a third aspect, there is provided a cDNA library obtained or obtainable by the method according to the second aspect of the present invention.

In a fourth aspect, there is provided a method of mapping one or more interactions between an RNA and an RNA binding protein, comprising the steps of: a) identifying an interaction between RNA and an RNA binding protein in a biological sample according to the method of the first aspect of the present invention; b) determining the location of the interaction in the genome; and preparing an RNA map of the one or more interactions

In a fifth aspect, there is provided a map obtained or obtainable by the method according to the fourth aspect.

In a sixth aspect, there is provided a method of mapping the effect of an RNA binding protein position on splicing regulation, comprising the steps of: a) identifying an interaction between an RNA and an RNA binding protein in a biological sample according to the method of the first aspect; and b) determining the positioning of one or more interactions in pre-RNA.

In a seventh aspect, there is provided a map obtained or obtainable by the method of the sixth aspect.

In an eighth aspect, there is provided a method for identifying an agent that modulates binding or association between an RNA an RNA binding protein of interest, comprising the steps of: a) determining an interaction between an RNA and an RNA binding protein in a biological sample according to the method of the first aspect in the presence and absence of the agent; and b) determining if the agent modulates the binding or association between the RNA and the RNA binding protein of interest, wherein a difference in the binding or association between the RNA and the RNA binding protein of interest in the presence of the agent is indicative that said agent modulates the binding or association.

In a ninth aspect, there is provided a method for identifying an agent that modulates binding or association between an RNA an RNA binding protein of interest, comprising the steps of: (a) preparing a map according to the fifth aspect in the presence and absence of the agent; and (b) determining if the agent modulates the binding or association between the RNA and the RNA binding protein of interest, wherein a difference in the map obtained in the presence of the agent as compared to the map obtained in the absence of the agent is indicative that said agent modulates the binding or association.

In a tenth aspect there is provided a method for identifying an agent that modulates splicing regulation, comprising the steps of: (a) preparing a map according to the seventh aspect in the presence and absence of the agent; and (b) determining if the agent modulates splicing regulation, wherein a difference in the map obtained in the presence of the agent as compared to the map obtained in the absence of the agent is indicative that said agent modulates splicing regulation.

### SUMMARY EMBODIMENTS OF THE INVENTION

In one embodiment, the covalent bond between the RNA and the RNA binding protein is created by cross-linking.

In one embodiment, the reverse transcription primer comprises a cleavable adapter.

In one embodiment, the reverse transcription primer comprises two inversely orientated adapter regions separated by a cleavable adapter.

In one embodiment, the cleavable adapter is cleavable by a restriction enzyme.

In one embodiment, said cleavable adapter additionally comprises one or more nucleotides of known or unknown sequence as an experiment identifier and/or to identify amplification duplicates.

In one embodiment, the one or more nucleotides of known or unknown sequence as an experiment identifier comprises at least two nucleotides.

In one embodiment, the one or more nucleotides of known or unknown sequence to identify amplification duplicates comprise at least three nucleotides.

In one embodiment, cDNA sequences that truncate at the same nucleotide in the genome and share the same one or more nucleotides of known or unknown sequence to identify amplification duplicates are eliminated from subsequent analysis.

In one embodiment, the circularised cDNA is linearised at the cleavable adapter.

In one embodiment, a primer complementary to at least a portion of the reverse transcription primer is hybridised thereto prior to linearisation.

In one embodiment, the cDNA is amplified by hybridising one or more primers that are complementary in sequence to at least a portion of the cleaved adapter.

In one embodiment, the nucleotide sequence of the amplified cDNA is determined up to the point that the cDNAs truncate at the crosslink site thereby providing individual nucleotide resolution of the crosslinking site.

In one embodiment, the nucleotide sequence of 5, 10, 20, 30, 40 or 50 or more of the nucleotides of the amplified cDNA up to the point that the cDNAs truncate at the crosslink site is determined.

In one embodiment, mapping of the interaction(s) is performed against the human genome.

In one embodiment, mapping of the interaction(s) is based on sequences that map to human nuclear chromosomes.

In one embodiment, amplification duplicates are excluded.

In one embodiment, the interaction(s) between RNA and an RNA binding protein are determined in replicate.

In one embodiment, reproducibility of crosslink nucleotides is determined by comparing all positions of crosslink nucleotides from the replicate(s).

In one embodiment, the positioning of one or more interactions is determined at an exon-intron boundary of alternative exons and/or flanking constitute exons and/or constitute exons.

In one embodiment, an exon-intron boundary of alternative exons and/or flanking constitute exons and/or constitute exons is identified using an array.

In one embodiment, the method described herein comprises the steps of: (a) assessing a first level of binding or association between the RNA and the RNA binding protein in a first cell, wherein said first cell has been contacted with the agent; (b) assessing a second level of binding or association between the RNA and the RNA binding protein in a second cell, wherein said second cell has not been contacted with the agent; and (c) comparing said first level of binding or association with said second level of binding or association, wherein a difference between said first level of binding or association and said second level of binding or association indicates an ability of said agent to modulate the association or binding between said RNA binding protein and said RNA.

### FIGURES

FIG. 1 iCLIP identifies hnRNP C cross-link nucleotides on RNAs.

(a) Schematic representation of the iCLIP protocol. After UV irradiation, the covalently linked RNA is co-immunoprecipitated with the RNA-binding protein (RBP) and ligated to an RNA adapter at the 3′ end. Proteinase K digestion leaves a covalently bound polypeptide fragment on the RNA that causes premature truncation of reverse transcription (RT) at the cross-link site. The red bar indicates the last nucleotide added during reverse transcription. Resulting cDNA molecules are circularized, linearized, PCR-amplified and subjected to high-throughput sequencing. The first nucleotides of each sequence contain the barcode followed by the nucleotide where cDNAs truncated during reverse transcription. (b) Reproducibility of cross-link nucleotide positions. Percentage of cross-link nucleotides with a given cDNA count that were identified in at least two (circles) or all three experiments (triangles) are shown. The percentage of reproduced cross-link nucleotides increased with the incidence of hnRNP C cross-linking (cDNA count). (c) Reproducibility of sequence composition at cross-link nucleotides. Frequencies of pentanucleotides overlapping with cross-link nucleotides are shown for the three replicate experiments (R²=0.9996, R²=0.9987 and R²=0.9996) with the sequence shown for the four most highly enriched pentanucleotides. 42% of cross-link nucleotides overlap with UUUUU in all three replicate experiments.

FIG. 2 The genomic location of hnRNP C cross-link nucleotides.

(a) Conversion of mapped iCLIP sequence reads into cDNA count values. Genomic sequence is shown above the color-coded positions of cDNA sequences from replicate experiments, preceded by the associated random barcode and the number of sequenced PCR duplicates (given in brackets). In the lower panel, a ‘cDNA count’ was assigned to the upstream ‘cross-link nucleotides’. Cross-link nucleotides within filtered clusters are highlighted in grey. The position of an alternative exon in CD55 mRNA is shown at the bottom. Modified image of the UCSC genome browser (human genome, version hg18, chromosome 1, nucleotides 205580308-205580373). * Due to space limitations, replicates 2 and 3 were merged into one lane. (b) Long-range spaced cross-link nucleotides flank the alternative exon in CD55 pre-mRNA. A distance of 165 nucleotides is marked by the red arrow with red shaded bars on either side representing a ten nucleotide surrounding interval. (c) Cross-link nucleotides are present along the entire length of CD55 pre-mRNA and accumulate around the alternative exon. Clustered cross-link nucleotides are indicated with grey lines. Annotation below shows position of exons in two alternative transcripts. (d) Global view of cross-link nucleotides on chromosome 11 (nucleotides 182200000-225000000). cDNA counts corresponding to positions in plus and minus strand transcripts are shown in blue and red, respectively. Gene annotations are given below. Cross-linking to individual genes and strand specificity are reproduced between replicates.

FIG. 3 hnRNP C binds uridine tracts with a defined spacing.

(a) Weblogo showing base frequencies of cross-link nucleotides and 20 nucleotides of surrounding genomic sequence. Positions 0 and 1 correspond to cross-link nucleotide and first position of cDNA sequence, respectively. For comparison, the background distribution of bases within transcribed regions is: U, 30.3%, A, 27.7%, G, 21.4% and C, 20.6%. (b) Length distribution of uridine tracts harboring cross-link nucleotides. The percentage of tracts of a certain length is given relative to all bound tracts. Panels compare all cross-link nucleotides (black) to those with a cDNA count of 2 or higher (grey, top), and length distribution of tracts within the transcriptome as control (bottom). (c) Positioning of cross-link nucleotides within uridine tracts. Positions were summarized over shorter (3-8 uridines, top) and longer tracts (9-15 uridines, bottom) aligned at their 3′ ends. Longer tracts contain two peaks at a defined spacing of 5-6 nucleotides (FIG. 12b). (d) Binding neighborhood of five-nucleotide uridine tracts (black). Occurrence of cross-link nucleotides at a given position is given as a fraction of all positions. Cross-link nucleotides within flanking uridine tracts of at least three uridines are shown in red, and those remaining in blue. (e) Long-range spacing of cross-link nucleotides. Distances to all downstream cross-link nucleotides were summarized (black). Uridine densities at the same distances are superimposed (red). Inlay shows an enlarged region of the graph. Increased occurrence of cross-link nucleotides coincided with peaks in uridine density at 165 and 300 nucleotides distance.

FIG. 4 The RNA map relates hnRNP particle positioning to splicing regulation.

(a) The RNA map of cross-link sites within regulated pre-mRNAs. Positioning of cross-link nucleotides was assessed at exon-intron boundaries of alternative (375 silenced, blue; 315 enhanced, red; 8571 control alternative exons, grey; regions of overlap are shown as lighter shades of blue/red) and flanking constitutive exons. “Occurrence (%)” indicates the percentage of exons that have at least one cross-link nucleotide within a given window. Black dots mark significant enrichment of regulated exons containing cross-link nucleotides within a given window relative to control alternative exons (p value<0.01 by Fisher's Exact test). Silenced alternative exons show strong enrichment of cross-link nucleotides proximal to the 3′ and the 5′ splice sites (3′SS and 5′SS). (b) The RNA map of hnRNP particles on regulated pre-mRNAs. Positioning of regions intervening cross-link nucleotides with defined 160-170 nucleotide spacing was analyzed as in FIG. 4a. Silenced alternative exons show incorporation of the entire regulated exon into hnRNP particles, whereas particle incorporation is confined to the preceding intron at enhanced alternative exons. (c) The RNA map of hnRNP particles at constitutive exons. Positioning of regions intervening the cross-link nucleotides with a spacing of 160-170 nucleotides was assessed at exon-intron boundaries of constitutive exons (29858 exons analyzed as in FIG. 4a). Splice sites show decreased incorporation into hnRNP particles.

FIG. 5 iCLIP data predict exons that are silenced by hnRNP C.

(a) Genomic location of hnRNP C cross-link nucleotides surrounding silenced exons that were predicted from iCLIP data. Five exons that are flanked by cross-link nucleotides with defined spacing and showed a significant increase in inclusion in the hnRNP C knockdown cells are depicted. cDNA counts corresponding to positions in plus and minus strand transcripts are shown in blue and red, respectively. Gene names and genomic sequence around cross-link nucleotides (highlighted by blue or red boxes indicating plus-strand or minus-strand location) are given above each panel. A distance of 165 nucleotides is marked by a red arrow with shaded bars on either side representing a ten nucleotide interval. Clustered cross-link nucleotides are highlighted in grey. A mutual exclusive exon in MTRF1 pre-mRNA is indicated by an asterisk. Images are based on the UCSC genome browser (human genome, version hg18; C12orf23, chromosome 12, nucleotides 105885065-105885394; MTRF1, chromosome 13, nucleotides 40734402-40734731; PRKAA1, chromosome 5, nucleotides 40810631-40810960; TBL1XR1, chromosome 3, nucleotides 178361247-178361576; ZNF195, chromosome 11, nucleotides 3347071-3347400). (b) Quantification of splicing changes of the alternative exons depicted in (a). RNA from hnRNP C knockdown (kd) and control (c) HeLa cells was analyzed using RT-PCR and capillary electrophoresis. Capillary electrophoresis image and signal quantification are shown for each exon. Quantified transcripts including (in) or excluding (ex) the regulated alternative exon are marked on the right. Average quantification values of exon inclusion (white) and exclusion (grey) are given as a fraction of summed values. Error bars represent standard deviation of three replicates. Change in exon inclusion and p values are given in Table 3. The asterisk indicates the PCR product for the RNA isoform of a mutually exclusive exon in MTRF1 pre-mRNA as depicted in (a). Its inclusion is strongly increased in hnRNP C knockdown cells consistent with our model that hnRNP C binding within the polypyrimidine tract leads to silencing of exons.

FIG. 6 A model of hnRNP C tetramer binding at silenced and enhanced alternative exons.

hnRNP C protein monomers are depicted in yellow with the RRM domains in grey. The schematic RNA molecule is shown to contact the RRM domains via uridine tracts and the bZLM domains via electrostatic interactions. Binding of the RRM domains on both sides of an alternative exon results in silencing of exon inclusion (blue), whereas tetramer binding to the preceding intron enhances exon inclusion (red).

FIG. 7 iCLIP experiments.

(a) Analysis of cross-linked hnRNP C-RNA complexes using denaturing gel electrophoresis and western blotting. Protein extracts were prepared from UV-cross-linked and control HeLa cells, and RNA was partially digested using low (+) or high (++) concentration of RNase. hnRNP C-RNA complexes were immuno-purified (IP) from cell extracts using an antibody against hnRNP C (a hnRNP C). The RNA adapter was ligated to the 3′ ends of RNAs before radioactively labeling the 5′ ends. Complexes were size-separated using denaturing gel electrophoresis and transferred to a nitrocellulose membrane. The upper panel shows an autoradiogram of this membrane. hnRNP C-RNA complexes shifting upwards from the size of the protein (40 kDa) can be observed (lane 2). The shift is less pronounced when high concentrations of RNase were used (lane 1). The radioactive signal disappears when hnRNP C is knocked down (lane 3 and 4), cells were not cross-linked (lane 5 and 6) or no antibody was used in IP (lane 7 and 8). The two red boxes (L1 and H1) mark regions of the membrane that were cut out for subsequent purification steps. The two lower panels show western blot analyses of protein extracts used as input for the IPs above. Antibody against hnRNP C visualizes knock-down efficiency, and antibody against GAPDH (α GAPDH) documents equal protein amounts in input extracts. (b) Analysis of PCR-amplified iCLIP cDNA libraries using denaturing gel electrophoresis. RNA recovered from membrane regions L1 and H1 (see above) was reverse transcribed and size-purified using denaturing gel electrophoresis (not shown). Two size fractions of cDNA (L2, 100-175 nt, and H2, 175-350 nt) were recovered, circularized, linearized and PCR-amplified. PCR products of different sizes can be observed according to different size combinations of input fractions (lane 1-4; L1 and H2 recovered from the protein membrane; L2 and H2 recovered from the cDNA gel). PCR products are absent when no antibody was used for the IP (lane 5-8) or no RNA was added to the reverse transcription reaction.

FIG. 8 Reproducibility analysis comparing replicate 1 with replicate 2.

Black bars show the number of cross-link nucleotides in replicate 1 that are reproduced in replicate 2 with a given offset. An offset of 0 nt indicates the number of cross-link nucleotides in replicate 1 that were reproduced by a crosslink nucleotide at exactly the same position in replicate 2. Negative or positive offset values indicate whether the reproducing position in replicate 2 is located upstream or downstream of the cross-link nucleotide in replicate 1, respectively. For example, the bar of height 5266 at offset +1 nt shows that 5266 cross-link nucleotides of replicate 1 were reproduced by a cross-link nucleotide 1 nt downstream in replicate 2. The orange curve depicts results of the same analysis upon randomization of cross-link nucleotide positions in replicate 2.

FIG. 9 Genomic location of hnRNP C cross-link nucleotides.

A pie chart depicting the fraction of cDNA sequences that map to different genomic regions (as given on the right; gene annotations based on UCSC hg18.known Gene).

FIG. 10 hnRNP C cross-linking to the regulatory element in c-myc mRNA.

A hnRNP C cross-link nucleotide locates to a seven nucleotide uridine tract within the c-myc mRNA. The corresponding genomic locus on chromosome 8 (128818008 to 128818059; modified UCSC genome browser image) surrounding the respective thymine tract (red) is shown. A cross-link nucleotide within the shown locus was only found in replicate 1. Binding of hnRNP C to this element within the internal ribosomal entry site (IRES) was shown to regulate alternative usage of an upstream start codon (CTG, green).

FIG. 11 Analyses of hnRNP C binding based on the clustered cross-link nucleotides dataset.

(a) Weblogo showing base frequencies of clustered cross-link nucleotides and 20 nucleotides of surrounding genomic sequence. Labeling as in FIG. 3a. Uridine represented 91% of cross-link nucleotides. (b) Length distribution of uridine tracts harboring clustered cross-link nucleotides. Analyses and labeling as in FIG. 3b. 83% of cross-link nucleotides were part of contiguous tracts of four or more uridines. (c) Positioning of clustered crosslink nucleotides within uridine tracts. Analyses and labeling as in FIG. 3c. Longer tracts contain two peaks at a defined spacing of 5-6 nucleotides. (d) Binding neighborhood of five nucleotide uridine tracts. Analysis and labeling as in FIG. 3d. Clustered cross-link nucleotides within 5 nt uridine tracts are commonly associated by flanking cross-link nucleotides again residing in uridin tracts. (e) Long-range spacing of clustered cross-link nucleotides. Distances to all downstream clustered cross-link nucleotides were summarized (black). Uridine densities at the same distances are superimposed (red). Inlay shows an enlarged region of the graph. Increased occurrence of clustered cross-link nucleotides coincided with peaks in uridine density at 165 and 300 nucleotides distance. (f) The RNA map of clustered cross-link nucleotides within regulated pre-mRNAs. Analysis and labeling as in FIG. 4a. Silenced alternative exons show strong enrichment of cross-link nucleotides proximal to the 3′ splice sites (3′SS).

FIG. 12 The dual pattern of hnRNP C cross-linking on uridine tracts.

(a) Fraction of cross-link nucleotides with a cDNA count of at least two on the third position from the 3′ end of uridine tracts of different lengths (as given below). With increasing tract length from 3 nt to 13 nt, cross-link nucleotides with a cDNA count of at least two represent an increasing proportion of all cross-link nucleotides (p value<10-5 by Wilcoxon rank sum test comparing tracts of 5 and 13 uridines). (b) Distribution of cross-link nucleotides over uridine tracts of different length. The number of cross-link nucleotides locating to each position is given as a fraction of all cross-link nucleotides locating to tracts of a given length. Crosslinking predominantly occurred on the third position from the 3′ end. In addition, tracts of more than eight uridines display a second peak at a constant distance of five or six nucleotides from the downstream peak.

FIG. 13 Analyses of differentially expressed transcripts in hnRNP C knockdown cells.

(a) Venn diagram depicting the significant overlap between differentially expressed transcripts (162 in total, including 115 decreased and 47 increased transcripts) and those that show a change in at least one alternative splicing event upon hnRNP C knockdown (1052 transcripts harboring a total of 1340 differentially spliced exons). 4.3% of the transcripts with at least one splicing change (45/1052) also showed a change in expression levels (compared to 0.7% [162/24571] when analysing all transcripts; p value=2.0×10-24 by to hypergeometric distribution). Vice versa, 27.7% of transcripts with changes in expression levels (45/162) harboured at least one differentially spliced exon (compared to 4.3% [1052/24571] of all transcripts; p value=2.0×10-24 by hypergeometric distribution). (b) Scatter plot comparing the change in expression level in the hnRNPC knockdown with the total number of hnRNP C cross link events per transcript. The red dashed lines indicate a change in transcript abundance by a factor of 2. We did not observe an apparent correlation between cross-linking and differential regulation (Pearson correlation coefficient 0.099 and 0.106 for decreased and increased transcripts, respectively).

FIG. 14 Western analysis of hnRNP C knockdown and control HeLa cells prepared for microarray and RT-PCR analyses.

Protein extracts from HeLa cells transfected with two different siRNAs (KD1 and KD2) were compared to control samples (Control). For each condition Western analysis is shown in triplicates (a, b and c). The upper panel was probed with an hnRNP C antibody (α hnRNP C), while the lower panel controls for loading using a GAPDH antibody (α GAPDH). Numbers on the left refer to the sizes of a protein standard in kDa.

FIG. 15 Quantification of splicing changes using RT-PCR and capillary electrophoresis.

(b) Quantification of alternative splicing in hnRNP C knockdown (kd) and control (c) HeLa cells. Capillary electrophoresis image and signal quantification are shown for each validated gene. Quantified transcripts including (in) or excluding (ex) the regulated alternative exon are marked on the right. Average quantification values of exon inclusion (white) and exclusion (grey) are given as a fraction of both. Error bars represent standard deviation of three replicate experiments. (a) and (b) show results for exons that are silenced and enhanced by hnRNP C, respectively. (c) Graph comparing the percent change values determined by quantitative PCR and splice-junction microarray analyses (Δ/values as determined with ASPIRES). Silenced (blue) and enhanced (red) alternative exons that were reproduced by quantitative PCR are shown as circles. Exons that displayed no change in quantitative PCR are depicted as black squares. Changes in 24 of 26 analyzed alternative exons could be reproduced.

### DEFINITIONS & DETAILED DESCRIPTION

### iCLIP

RNA-protein interactions are pivotal in fundamental cellular processes, such as translation, RNA splicing, regulation of key decisions in early development, and infection by RNA viruses. However, in spite of the central importance of these interactions, few in vivo approaches are available to analyze them. There is described herein a method to precisely identify RNA-protein interactions in vivo. Accordingly, the method can be used to identify the precise nucleotide sequence (eg. the individual nucleotide sequence) at which the RNA-protein interaction(s) occur in vivo. In one embodiment, the method comprises the steps of: a) contacting the biological sample with an agent that creates a covalent bond between the RNA and the RNA binding protein; b) fragmenting said RNA; c) ligating a first adapter to the fragmented RNA; d) hybridising a reverse transcription primer to said first adapter and reverse transcribing said cross-linked RNA; e) circularising the transcribed cDNA; f) linearising the circularised cDNA; and g) determining the sequence of one or more of the cDNAs. In one embodiment, following cross-linking of RNA and protein, the covalently linked RNA/RNA-binding protein is obtained and an RNA adapter is ligated to the 3′ end of the RNA. In one embodiment, a protease is used to digest the RNA binding protein, thereby leaving a covalently bound polypeptide (eg. a covalently bound polypeptide fragment) on the RNA. In one embodiment, following reverse transcription, the polypeptide causes premature truncation of cDNAs at the crosslink site. cDNA molecules may then be circularised to attach an adapter sequence and optionally a barcode to the truncated end, linearised, amplified and optionally subjected to DNA sequencing. The first nucleotide of each sequence contains the barcode followed by the nucleotide where cDNAs truncated during reverse transcription. Further aspects and embodiments of the method are described herein.

### RNA Binding Protein

RNA-binding proteins have a role in a wide variety of cellular and developmental functions. For example, they participate in RNA processing, editing, transport, localization, stabilization, and the posttranscriptional control of mRNAs. The RNA binding activity of these proteins is mediated by specific RNA-binding domains contained within the proteins. A variety of conserved RNA binding motifs have been defined through comparisons of amino acid homologies and structural similarities within these RNA-binding domains. These motifs include the RNP motif, an arginine-rich motif, the zinc-finger motif, the Y-box, the KH motif, and the double-stranded RNA-binding domain (dsRBD), all of which are characterized by specific consensus sequences (Burd, C. G. and Dreyfuss, G. (1994) Science 265:615-621).

As used herein, the term “RNA binding protein” refers to any peptide, polypeptide, or peptide-containing substance or complex that specifically interacts with a RNA strand or RNA strands. The RNA binding protein may be a complex of two or more individual molecules, which may be the same (eg. a homodimer) or different (eg. a heterodimer). The RNA binding protein may be sequence specific such that it binds to a specific sequence or family of specific sequences—such as a sequence motif—that may show a high degree of sequence identity with each other with greater affinity than to unrelated sequences. Alternatively, the RNA binding protein may be non-sequence specific such that it binds to a plurality of unrelated sequences.

### Interaction

As used herein, “an interaction between an RNA and an RNA binding protein” refers to a physical association—such as a covalent association between one or more RNA molecules and one or more RNA binding proteins, or one or more RNA binding protein complexes made up of one or more RNA binding proteins.

### Biological Sample

The methods described herein are suitable for identifying an interaction between RNA and an RNA binding protein in a biological sample (eg. in vitro or in vivo). The term “biological sample” as used herein, has its natural meaning. The sample may be any physical entity comprising an RNA and/or an RNA binding protein that is or is capable of being cross-linked. The sample may be or may be derived from biological material.

The sample may be or may be derived from one of more entities—such as one or more cells (eg. mammalian or human cells) or one or more tissue samples. The entities may be or may be derived from any entities in which RNA and/or an RNA binding protein is present. The sample may be or may be derived from one or more isolated cells or one or more isolated tissue samples. The sample may be or may be derived from living cells and/or dead cells. The sample may be or may be derived from diseased and/or non-diseased subjects. The sample may be or may be derived from a subject that is suspected to be suffering from a disease. The sample may be or may be derived from viable or non-viable patient material. The sample may be or may be derived from combinations thereof.

The sample may be or may be derived from a cell culture, a cell line, a cell extract, a cell lysate, whole tissue, a tissue extract, a tissue sample—such as a biopsy, a whole organ, a tumor, a tumor cell, a cell mass, diseased tissue, tumor cell extract, a pre-cancerous lesion, a polyp, a cyst and/or a combination thereof.

Cells comprising the biological sample may be a suspension cells, adherent cells, transformed cells, tissue culture cells or primary cell lines.

The biological sample may disrupted, disaggregated, homogenized, or lysed by any technique known in the art. For example, the biological sample may be made into a single-cell suspension using a nylon filter or mesh. Cells or tissue comprising the biological sample may be adhered to a substrate such as a chip, a slide, a dish or the like.

In an embodiment of the method described herein, the cells—such as HeLa cells—are grown and then subjected to one or more cross-linking agents.

### Covalent Bond

The method described herein comprises the step of contacting the biological sample with an agent that creates a covalent bond between RNA and a RNA binding protein. Suitably, the biological sample is contacted with an agent that cross links the RNA binding protein to RNA.

Cross-linking agents—such as formaldehyde—may be used to cross link one or more proteins—such as one or more RNA binding proteins—to nucleic acid—such as RNA. Other cross-linking agents may also be used in accordance with the present invention, including those cross-linking agents that directly cross link nucleotide sequences. Examples of agents that cross-link nucleic acid include, but are not limited to, UV light, mitomycin C, nitrogen mustard, melphalan, 1,3-butadiene diepoxide, cis diaminedichloroplatinum(II) and cyclophosphamide.

In one embodiment, the crosslinking agent is UV light.

Following cross linking, cells may be precipitated by centrifugation and shock-frozen on dry ice.

### Fragmenting

In a further step of the method described herein, RNA is fragmented. The nucleic acid may be randomly fragmented. Various methods of fragmenting nucleic acids will be known to those of skill in the art. These methods may be chemical and/or physical in nature.

In one embodiment, fragmentation may comprise partial degradation with a RNAse or partial depurination with acid followed by heating. Physical fragmentation methods may involve subjecting the nucleic acid to a high shear rate. High shear rates may be produced by moving nucleic acid through a chamber or channel with pits or spikes, or forcing the nucleic sample through a restricted size flow passage, e.g. an aperture having a cross sectional dimension in the micron or submicron scale.

Other methods of fragmentation include the use of radical-generating coordination complexes or with a syringe-operated silica micro-column or through the use of heat or ion-mediated hydrolysis.

In one embodiment, RNA is fragmented using partial RNase digestion.

Suitably, the fragmented RNAs may be 40 to 80 nucleotides in length.

Following the fragmentation, cells may be precipitated and the supernatant collected for subsequent isolation of fragmented nucleic acid. The supernatant may be added to and incubated with, for example, Dynabeads for a suitable amount of time, which are then washed.

### First Adapter

In a further step of the method described herein, a first adapter—such as a first RNA adapter—may be ligated to the fragmented RNA. Suitably, the 3′ end of the fragmented nucleic acid is dephosphorylated prior to first adapter ligation.

As used herein, the term “adapter” may be used interchangeably with the term “linker” and their meanings are intended to be the same ie. an oligonucleotide that is joined to nucleic acid. In one embodiment, the adapter is suitable for directional ligation. In one embodiment, the adapter (eg. the first adapter) does not comprise a polyA tail.

The first adapter may be ligated to one or both ends of the nucleic acid fragments to facilitate the hybridisation of a primer (eg. an RT-PCR primer) and/or cDNA synthesis. Suitably, the first adapter is ligated at the 3′ end of the nucleic acid (RNA) fragments. In one embodiment, an adapter is not ligated at the 5′ end of the nucleic acid (RNA) fragments.

The first adapter may comprise, consist or consist essentially of RNA and/or DNA or a derivative thereof. In one embodiment, the first adapter comprises, consists or consist essentially of RNA and it may comprise the sequence:

### 5′-UGAGAUCGGAAGAGCGGTTCAG-3′

Suitably, the cross-linked RNA with the first adapter ligated thereto is isolated using methods known in the art.

The RNA binding protein that is bound to the RNA may be digested. This may be achieved using a suitable protease. In one embodiment, the protease that is used is proteinase K. According to this embodiment, a covalently bound polypeptide—such as a covalently bound polypeptide fragment—remains bound to the RNA at the cross-linked site.

In one embodiment, the first adapter is ligated to the 3′ end of the RNA and the protease is then used to digest the RNA binding protein. In another embodiment, the protease is used to digest the RNA binding protein and the first adapter is then ligated to the 3′ end of the RNA.

The use of the first adapter may provide a sequence to which a primer—such as a reverse transcription primer—may hybridise. The first adapter may be fully or partially complementary to a primer. If the first adapter is partially complementary to the primer then the first adapter should still specifically hybridise to the primer.

### Reverse Transcription Primer

In a further step of the method described herein, a reverse transcription primer comprising a cleavable adapter is hybridised to said first adapter to reverse transcribe the RNA ligated to the first adapter. Suitably the cleavable adapter is cleavable or cleaved at a defined sequence—such as a sequence that is recognised by a restriction enzyme. In one embodiment, the cleavable adapter comprises two inversely oriented adapter regions with a cleavable sequence (eg. a BamHI restriction enzyme site) separating the two inversely oriented adapter regions.

The reverse transcription primer may comprise one or more defined or random nucleic acid sequences that function as a barcode. In one embodiment, this barcode (eg. a defined sequence barcode) may be used to analyse, determine or quantify the individual cDNA molecules when analysing the final data from the method described herein. In another embodiment, the barcode (eg. a random sequence barcode) may be used to separate sequences mapping to the same crosslink nucleotide which are an artefact of amplification from those that are unique cDNA products. The barcode sequences may be of any suitable length and sequence.

The reverse transcription primer may be fully or partially complementary to the first adapter. If the primer is partially complementary to the first adapter then the primer should still specifically hybridise to the first adapter. Suitably the primer can self-circularise. Suitably the primer can self-circularise and cannot serve as a template in a subsequent amplification reaction.

Following reverse transcription from the 3′ end of the reverse transcription primer, the reverse transcriptase truncates at the cross-linked site where the covalently bound polypeptide is bound to the RNA.

RNA may be removed following reverse transcription. cDNAs may be precipitated following reverse transcription.

In one embodiment, the reverse transcription primer comprises, consists or consist essentially of the one or more of the following sequences:

5′-AGATCGGAAGAGCGTCGTGGATCCTGAACCGCTC-3′;
5′-NNNAGATCGGAAGAGCGTCGTGGATCCTGAACCGCTC-3′;
5′-NNNCAAGATCGGAAGAGCGTCGTGGATCCTGAACCGCTC-3′;
5′-AGATCGGAAGAGCGTCGTGGATCCTGAACCGC-3′;
5′-NNNAGATCGGAAGAGCGTCGTGGATCCTGAACCGC-3′;
5′-NNNGAAGATCGGAAGAGCGTCGTGGATCCTGAACCGC-3′;
5′- AGATCGGAAGAGCGTCGTGGATCCTGAACCGCTC-3′;
5′- NNNAGATCGGAAGAGCGTCGTGGATCCTGAACCGCTC-3′;
5′- NNNTGAGATCGGAAGAGCGTCGTGGATCCTGAACCGCTC-3′;

wherein NNN represents a 3-nucleotide random sequence (random barcode that marks unique cDNA molecules); and the bold nucleotides represent a 2-nucleotide defined sequence barcode (sequence that marks the primer used for RT, and allows multiplexing of multiple RT reactions in a single sequencing reaction). The length of the random barcodes can be increased to increase the range quantitative analysis, and the length of defined barcode can be increased to allow multiplexing of a larger number of samples. According to this embodiment, the 2-nucleotide barcode may be used as an experiment identifier and the 3-nucleotide random barcode may be used to identify amplification duplicates.

Suitably, the transcribed cDNAs are separated from the cross-linked RNA/RNA adapter sequence.

In a further aspect, there is provided a nucleotide sequence hybrid comprising, consisting or consisting essentially of: (a) an RNA sequence with a polypeptide bound at a crosslinked nucleotide and an RNA adapter at the 3′ end thereof; and (b) a reverse transcription primer comprising a cleavable adapter hybridised to at least a portion of the RNA adapter.

In a further aspect, there is provided a nucleotide sequence hybrid comprising, consisting or consisting essentially of: (a) an RNA sequence with a polypeptide bound at a crosslinked nucleotide and an RNA adapter at the 3′ end thereof; and (b) a reverse transcription primer comprising a cleavable adapter hybridised to at least a portion of the RNA adapter and a cDNA sequence that is complimentary to the RNA sequence juxtaposed between the crosslinked nucleotide and the RNA adapter.

### Circularising

In a further step of the method described herein, the transcribed cDNA with the reverse transcription primer and optionally the bar code may be circularised, using for example, a DNA ligase.

In a further aspect, there is provided a circularised nucleotide sequence comprising, consisting or consisting essentially of a reverse transcription primer comprising a cleavable adapter and a cDNA sequence that is complimentary to an RNA sequence adjacent to a crosslinked nucleotide.

### Linearising

In a further step of the method described herein, the circularised cDNA is linearised. Suitably, the circularised DNA is linearised at a different position compared to where the transcribed cDNA is circularised. Suitably, the circularised DNA is linearised at the cleavable adapter.

A linearisation primer may be hybridised to at least a portion of the cleavable adapter prior to linearisation at the cleavable adapter. In one embodiment, the linearisation primer comprises the sequence:

5′-GTTCAGGATCCACGACGCTCTTCAAAA-3′

Following cleavage, a linearised nucleotide sequence may result comprising, consisting or consisting essentially of: (a) a cDNA sequence complimentary to at least a portion of RNA that is adjacent a covalent bond formed between RNA and a RNA binding protein; (b) a cleaved adapter, wherein each of the 5′ and 3′ ends of the cDNA sequence comprise at least a portion of said adapter; and optionally (c) a bar code juxtaposed between the 5′ end of the cDNA sequence of the 3′ end of the cleaved adapter that is located at the 5′ end of the cDNA sequence. Optionally, an amplification primer may be hybridised to the cleaved adapter at the 5′ end of the linearised sequence.

### Determining the Sequence

In a further step of the method described herein, the sequence of some or all of the cDNA(s) is determined. Suitably, the cDNA is first amplified prior to sequencing. In one embodiment, the primers are complementary to each end of the cleaved adapter.

Amplification of nucleic acid—such as DNA or cDNA—may be performed using a number of different methods that are known in the art. For example, nucleic acid may be amplified using the polymerase chain reaction, ligation mediated PCR, Qb replicase amplification, the ligase chain reaction, the self-sustained sequence replication system and strand displacement amplification. Commonly, nucleic acid is amplified using PCR as described in U.S. Pat. No. 4,683,195, U.S. Pat. No. 4,683,202, and U.S. Pat. No. 4,965,188.

In one embodiment, the primers may be complementary to each end of the cleaved adapter. In another embodiment, the primers may be PCR primers that are complementary to each end of the cleaved adapter. The primers may comprise the sequence:

5′-CAAGCAGAAGACGGCATACGAGATCGGTCTCGGCATTCCTGC
TGAACCGCTCTTCCGATCT-3′;
5′-AATGATACGGCGACCACCGAGATCTACACTCTTTCCCTACAC
GACGCTCTTCCGATCT-3′

The cDNA that is sequenced may be in the form a cDNA library representing a collection of fragments of DNA that represent the sequence information obtained by the methods of the present invention. The members of the library may be in the form of the circularised or linearised sequences described herein, or the linearised sequences may be inserted by well known molecular techniques into self-replicating units—such as cloning vectors. Each DNA fragment is therefore represented as part of an individual molecule, which may be reproduced in a single bacterial colony or bacteriophage plaque.

Suitably, the sequences are determined using high-throughput sequencing of iCLIP cDNA libraries which may be derived from replicate experiments. Suitably, the sequence reads will include one or more of the barcodes described herein. Examples of high-throughput sequencing approaches are described in K Y. Chan, Mutation Research 573 (2005) 13-40 and include, but are not limited to, near-term sequencing approaches—such as cycle-extension approaches, polymerase reading approaches and exonuclease sequencing, revolutionary sequencing approaches—such as DNA scanning and nanopore sequencing and direct linear analysis. Specific examples of current high-throughput sequencing methods are pyrosequencing, Solexa sequencung, Agencourt SOLiD sequencing and MS-PET sequencing.

The length of the sequence reads may vary. For some embodiments, it may be desirable to read 50 or more of the nucleotides. For some embodiments, it may be desirable to read 40, 30, 20, 10 or less nucleotides. Advantageously, the sequence reads will provide nucleotide sequence information up to the point that the cDNAs truncate at the crosslink site thereby providing individual nucleotide resolution of the crosslinking site. Suitably, the sequence 3′ to the crosslink site is read. Accordingly, in one embodiment, the nucleotide sequence of 5, 10, 20, 30, 40 or 50 or more of the nucleotides of the amplified cDNA up to the point that the cDNAs truncate at the crosslink site is determined. In a further embodiment, the nucleotide sequence of 5, 10, 20, 30, 40 or 50 or more of the nucleotides of the amplified cDNA up to the point that the cDNAs truncate at the 3′ side of the crosslink site is determined.

In a further aspect of the present invention, there is provided a method for identifying an interaction between an RNA and an RNA binding protein in a biological sample, comprising the steps of: a) contacting the biological sample with an agent that creates a covalent bond between the RNA and the RNA binding protein; b) fragmenting said RNA; c) ligating a first adapter to 3′ end of the fragmented RNA; d) digesting the crosslinked RNA binding protein to leave a polypeptide at the crosslink site; e) hybridising a reverse transcription primer comprising a cleavable adapter to said first adapter and reverse transcribing said cross-linked RNA into cDNA; f) circularising the transcribed cDNA; g) linearising the circularised cDNA at the cleavable adapter; h) amplifying the cDNA; and g) determining the sequence of the cDNA.

### Nucleic Acid

The term “nucleic acid” as used herein has its conventional meaning as used in the art and refers to a string of at least two base-sugar-phosphate combinations.

The term may include, deoxyribonucleic acid (DNA) and ribonucleic acid (RNA).

RNA may be in the form of a tRNA (transfer RNA), snRNA (small nuclear RNA), rRNA (ribosomal RNA), mRNA (messenger RNA), anti-sense RNA, small inhibitory RNA (siRNA), micro RNA (mRNA) and ribozymes. In one embodiment, the RNA is not synthetically polyadenyalted RNA.

DNA may be in form plasmid DNA, viral DNA, linear DNA, or chromosomal DNA or derivatives of these groups.

The nucleic acid may be double-stranded or single-stranded whether representing the sense or antisense strand or combinations thereof or even triple, or quadruple stranded.

The nucleic acid may be of genomic, synthetic or recombinant origin.

The term also includes, in one embodiment, artificial nucleic acids that may contain other types of backbones but the same bases. Examples of artificial nucleic acids are PNAs (peptide nucleic acids), phosphorothioates, and other variants of the phosphate backbone of native nucleic acids. PNA contain peptide backbones and nucleotide bases, and are able to bind both DNA and RNA molecules. The use of phosphothiorate nucleic acids and PNA are known to those skilled in the art, and are described in, for example, Neilsen P E, Curr Opin Struct Biol 9:353-57; and Raz N K et al. Biochem. Biophys Res Commun. 297:1075-84. For the purposes of the present invention, it is to be understood that the nucleotide sequences described herein may be modified by any method available in the art. Such modifications may be carried out in order to enhance the in vivo activity or life span of nucleotide sequences of the present invention.

### Hybridisation

The term “hybridisation” as used herein includes “the process by which a strand of nucleic acid joins with a complementary strand through base pairing” as well as the process of amplification as carried out in, for example, polymerase chain reaction (PCR) technologies.

Nucleotide sequences capable of selective hybridisation will be generally be at least 75%, preferably at least 85 or 90% and more preferably at least 95% or 98% homologous to the corresponding complementary nucleotide sequence over a region of at least 20, preferably at least 25 or 30, for instance at least 40, 60 or 100 or more contiguous nucleotides.

“Specific hybridisation” refers to the binding, duplexing, or hybridising of a molecule only to a particular nucleotide sequence under stringent conditions (e.g. 65° C. and 0.1×SSC (1×SSC=0.15 M NaCl, 0.015 M Na-citrate pH 7.0)). Stringent conditions are conditions under which a probe will hybridise to its target sequence, but to no other sequences. Stringent conditions are sequence-dependent and are different in different circumstances. Longer sequences hybridise specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to a target sequence hybridise to the target sequence at equilibrium. (As the target sequences are generally present in excess, at Tm, 50% of the probes are occupied at equilibrium). Typically, stringent conditions include a salt concentration of at least about 0.01 to 1.0 M Na ion concentration (or other salts) at pH 7.0 to 8.3 and the temperature is at least about 30° C. for short probes. Stringent conditions can also be achieved with the addition of destabilising agents—such as formamide or tetraalkyl ammonium salts.

### Homologues

The nucleotide sequences described herein have a degree of sequence identity or sequence homology. The term “homologue” may be equated with “identity”.

A homologous sequence is taken to include a nucleotide sequence which may be at least 50%, preferably at least 55%, such as at least 60%, for example at least 65%, at least 70%, at least 75%, at least 80%, at least 85%, at least 90%, at least 95%, at least 96%, at least 97%, at least 98% or at least 99%, identical to the subject sequence.

Sequence identity comparisons can be conducted by eye, or more usually, with the aid of readily available sequence comparison programs. Suitable computer programs for carrying out alignments include, but are not limited to, Vector NTI (Invitrogen Corp.) and the ClustalV, ClustalW and ClustalW2 programs. A selection of different alignment tools are available from the ExPASy Proteomics server at www.expasv.org. Another example of software that can perform sequence alignment is BLAST (Basic Local Alignment Search Tool), which is available from the webpage of National Center for Biotechnology Information and which was first described in Altschul et al. (1990) J. Mol. Biol. 215; 403-410.

Once the software has produced an alignment, it is possible to calculate % similarity and % sequence identity. The software typically does this as part of the sequence comparison and generates a numerical result.

### Mapping

RNA maps may be prepared to map crosslink sites relative to alternative exons in vivo. Such RNA maps be of use in determining the positioning of RNA binding proteins on pre-mRNAs in vivo and may provide insights into RNA splicing and the role of splicing regulation in tissue-specific functions.

In order to analyse the impact of RNA binding protein positioning on splicing regulation, the positioning of RNA binding protein crosslink sites can be assessed on RNA at, for example, exon-intron boundaries of alternative exons and flanking constitutive exons. For the preparation of RNA maps, regions may be divided into non-overlapping windows of stretches of nucleotides. For each window, the number of crosslink nucleotides may be counted as 1 if at least one crosslink nucleotide resided within the window. Thus, the resulting occurrence value reflects the number of exons with at least one crosslink nucleotide within the window. Percentages may be calculated by dividing the number of exons that have at least one crosslink nucleotide within a given window by the total number of exons analysed at this window.

Methods for the preparation of RNA maps to map crosslink sites relative to alternative exons are described herein.

### Array

Aspects of the invention may comprise the use of microarray analysis. In one embodiment, the array is a splicing array²⁴. For some embodiments, it may be appropriate to use splice-junction microarrays that allow for the monitoring of exon-exon junctions and/or individual exons and the like. The positioning of the crosslink sites at exon-exon boundaries and/or exon-intron boundaries and/or flanking constitutive exons and/or exons and/or introns and/or may then be analysed in order to prepare RNA maps.

An “array” is an intentionally created collection of nucleic acids which can be prepared either synthetically or biosynthetically. and screened for biological activity in a variety of different formats (e.g., libraries of soluble molecules; and libraries of oligos tethered to resin beads, silica chips, or other solid supports). Additionally, the term “array” includes those libraries of nucleic acids which can be prepared by spotting nucleic acids of essentially any length (e.g., from 1 to about 1000 nucleotide monomers in length) onto a substrate.

Array technology and the various techniques and applications associated with it is described generally in numerous textbooks and documents. These include Lemieux et al, 1998, Molecular Breeding 4, 277-289, Schena and Davis. Parallel Analysis with Biological Chips, in PCR Methods Manual (eds. M. Innis, D. Gelfand, J. Sninsky), Schena and Davis, 1999, Genes, Genomes and Chips. In DNA Microarrays: A Practical Approach (ed. M. Schena), Oxford University Press, Oxford, UK, 1999), and Eakins and Chu, 1999, Trends in Biotechnology, 17, 217-218.

Arrays are available for the analysis of splicing—such as alternative splicing—and may be of use in accordance with the present invention. For example, Hu et al. (Genome Research 11:1237-1245, 2001) disclose the use of DNA microarrays for the purpose of detecting alternative splicing in different rat tissues. Their technology relies on sequence information derived from comparing mature mRNAs only, and does not require knowledge of exon-exon splice junctions nor any intronic or other genomic sequence. Each gene of the microarray is represented by a set of twenty pairs of 25-mer oligonucleotides designed from EST and cDNA sequence information. Alternative splice variants are detected by virtue of the loss of hybridization signal from one or more of the probes in one tissue type versus another.

Shoemaker et al. (Nature 409: 922-927, 2001) discloses a method for experimentally confirming the existence of exons predicted by bioinformatics algorithms, then refining knowledge of the structure of the confirmed exons. The method involves construction and sequential use of two types of DNA microarrays. The first array comprises oligonucleotide probes of predicted exons. This ‘exon-array’ is used to experimentally confirm exons predicted from bioinformatics algorithms. Hybridization of a given probe to mRNA from a particular tissue type indicates that the exon is ‘authentic’. Exons are grouped into genes based on observations of coordinated expression of adjacent exons in a variety of tissues.

WO01/57252 discloses a “single exon microarray” for experimentally confirming exons predicted from genomic sequence data using bioinformatics algorithms. This method is similar to Shoemaker et al. discussed above. Oligonucleotide probes that make up the single exon microarray are comprised of predicted exonic sequences derived from genomic DNA. The array is hybridized with mRNA from different tissues, and based on the intensity of the hybridization signal of adjacent exons, conclusions are drawn about the different RNA isoforms present in different tissues. Identification of spliced and unspliced transcripts is made inferentially by comparison of fluorescence intensities of adjacent probes in different tissues.

As used herein, an “intron” is as generally understood in the art—a genomic nucleic acid sequence that is removed during mRNA splicing in the generation of a particular spliced mRNA variant. In other words, within one spliced variant of a gene, an intron is removed by mRNA splicing.

As used herein, an “exon” is as generally understood in the art—a genomic nucleic acid sequence that is retained during mRNA splicing in the generation of a particular spliced mRNA variant. In other words, within one spliced variant of a gene, an exon is retained by mRNA splicing.

It is understood that “intron” and “exon” are relative with respect to a particular mRNA spliced variant, and that an exon of one spliced variant may be an intron of another, and vice versa. However, within one spliced variant, an “intron” cannot be an “exon” and vice versa.

A “splice junction” is as generally understood in the art—a junction between two exons within a particular spliced variant of a gene. The splice junction is a product of mRNA splicing, and the contiguous sequence bridging the splice junction (e.g., a contiguous sequence extending from the 3′ end of a first exon, across the junction, and to the 5′ end of a second exon) is not present in the corresponding genomic DNA.

A “splice site” is as generally understood in the art—a site between an exon and an adjacent intron in unspliced mRNA, and can either be at the 5′ end an intron, or the 3′ end of an intron.

“Constitutively spliced exon” is as generally understood in the art—an exon that is present in all mRNA spliced variants of a selected gene.

### Assay

A further aspect of the present invention relates to a method for identifying an agent that modulates binding or association between RNA and an RNA binding protein. Accordingly, this aspect of the present invention may be used to identify inhibitors (eg. antagonists) or stimulators (eg. agonists) of one or more RNA/RNA binding protein interactions.

The screening assay may be performed in a cell-based system or a cell-free system. Cell-based assays may utilise cells that normally express the RNA binding protein. In an alternate embodiment, the cell-based assay may involve recombinant host cells expressing the RNA binding protein.

Agents to be tested could be directly applied to a cell or added to the growth medium. Substances that could be tested in this way include organic and inorganic molecules of any type—such as naturally occurring organic molecules, synthetic organic molecules, or crude extracts from micro-organisms and the like. Cells could be exposed to a range of concentrations of the substance, or substances to determine their impact on one or more RNA/RNA binding protein interactions. Agents may be introduced into a cell via cloned DNA. Accordingly, the cell may be transformed with a library of DNAs, each one of which encodes a different peptide or protein—such as an RNA binding protein. The peptides or proteins could be artificial, generated from random sequence, or could be derived from naturally occurring proteins (as in a cDNA library). Using cloned DNA libraries, a very large number of sequences could be screened.

In one embodiment, the method comprises (a) determining an interaction between an RNA and an RNA binding protein in a biological sample according to the method described herein in the presence and absence of an agent; and (b) determining if the agent modulates the binding or association between the RNA and the RNA binding protein of interest, wherein a difference in the binding or association between the RNA and the RNA binding protein of interest in the presence of the agent is indicative that said agent modulates the binding or association.

Suitably, said method comprises the steps of: (a) assessing binding or association between the RNA and the RNA binding protein in a first cell, wherein said first cell has been contacted with the agent; (b) assessing binding or association between the RNA and the RNA binding protein in a second cell, wherein said second cell has not been contacted with the agent; and (c) comparing said binding or association in the presence of the agent with said level of binding or association in the presence of the agent, wherein a difference between the binding or association in the presence of the agent and the level of binding or association in the absence of the agent indicates an ability of said agent to modulate the association or binding between said RNA binding protein and said RNA.

### Kits

The materials for use in the methods described herein are suited for preparation of kits. Such a kit may comprise containers, each with one or more of the various reagents (typically in concentrated form) utilised in the methods described herein, including, for example, a ligase, a protease, a reverse transcriptase, a first adapter, a reverse transcription primer, and optionally amplification primers and amplification reagents. Oligonucleotides may be provided in containers which can be in any form, e.g., lyophilized, or in solution (e.g., a distilled water or buffered solution), etc. A set of instructions will also typically be included.

### General Recombinant DNA Methodology Techniques

The present invention employs, unless otherwise indicated, conventional techniques of molecular biology, microbiology and recombinant DNA, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press.

### Further Aspects

Further aspects and embodiments of the present invention are presented in the following numbered paragraphs:

1. A method for identifying an interaction between an RNA and an RNA binding protein in a biological sample, comprising the steps of:

a) contacting the biological sample with an agent that creates a covalent bond between the RNA and the RNA binding protein;
b) fragmenting said RNA;
c) ligating a first adapter to the fragmented RNA;
d) hybridising a reverse transcription primer to said first adapter and reverse transcribing said cross-linked RNA into cDNA;
e) circularising the transcribed cDNA;
f) linearising the circularised cDNA; and
g) determining the sequence of one or more of the cDNAs.

2. The method of paragraph 1, wherein the covalent bond between the RNA and the RNA binding protein is created by cross-linking.

3. The method according to paragraph 1 or paragraph 2, wherein the reverse transcription primer comprises a cleavable adapter.

4. The method according to paragraph 3, wherein the reverse transcription primer comprises two inversely orientated adapter regions separated by a cleavable adapter.

5. The method according to paragraph 3 or paragraph 4, wherein the cleavable adapter is cleavable by a restriction enzyme.

6. The method according to any of paragraphs 3 to 5, wherein said cleavable adapter additionally comprises one or more nucleotides of known or unknown sequence as an experiment identifier and/or to identify amplification duplicates.

7. The method according to paragraph 6, wherein the one or more nucleotides of known or unknown sequence as an experiment identifier comprises at least two nucleotides.

8. The method according to paragraph 6 or paragraph 7, wherein the one or more nucleotides of known or unknown sequence to identify amplification duplicates comprise at least three nucleotides.

9. The method according to any of the preceding paragraphs, wherein cDNA sequences that truncate at the same nucleotide in the genome and share the same one or more nucleotides of known or unknown sequence to identify amplification duplicates are eliminated from subsequent analysis.

10. The method according to any of paragraphs 3 to 9, wherein the circularised cDNA is linearised at the cleavable adapter.

11. The method according to any of the preceding paragraphs, wherein a primer complementary to at least a portion of the reverse transcription primer is hybridised thereto prior to linearisation.

12. The method according to any of the preceding paragraphs, wherein the cDNA is amplified by hybridising one or more primers that are complementary in sequence to at least a portion of the cleaved adapter.

13. The method according to any of the preceding paragraphs, wherein the nucleotide sequence of the amplified cDNA is determined up to the point that the cDNAs truncate at the crosslink site thereby providing individual nucleotide resolution of the crosslinking site.

14. The method according to paragraph 13, wherein the nucleotide sequence of 5, 10, 20, 30, 40 or 50 or more of the nucleotides of the amplified cDNA up to the point that the cDNAs truncate at the crosslink site is determined.

15. A method for preparing a cDNA library representative of one or more interactions between an RNA and an RNA binding protein, comprising the steps of:

a) contacting the biological sample with an agent that creates a covalent bond between the RNA and the RNA binding protein;
b) fragmenting said RNA;
c) ligating a first adapter to the fragmented RNA;
d) hybridising a reverse transcription primer to said first adapter and reverse transcribing said cross-linked RNA;
e) circularising the transcribed cDNA;
f) optionally linearising the circularised cDNA; and
g) optionally sub-cloning the linearised cDNA into a vector.

16. A method of mapping one or more interactions between an RNA and an RNA binding protein, comprising the steps of:

a) identifying an interaction between an RNA and an RNA binding protein in a biological sample according to the method of any of paragraphs 1 to 14; and
b) determining the location of the interaction in the genome.

17. The method according to paragraph 16, wherein mapping of the interaction(s) is performed against the human genome to determine the position of crosslink nucleotides.

18. The method according to paragraph 16 or 17, wherein mapping of the interaction(s) is based on sequences that map to human nuclear chromosomes.

19. The method according to any of paragraphs 16 to 18, wherein amplification duplicates are excluded.

20. The method according to any of paragraphs 16 to 19, wherein the interaction(s) between RNA and an RNA binding protein are determined in replicate.

21. The method according to paragraph 20, wherein reproducibility of crosslink nucleotides is determined by comparing all positions of crosslink nucleotides from the replicate(s).

22. A method of mapping the effect of an RNA binding protein position on splicing regulation, comprising the steps of:

a) identifying an interaction between an RNA and an RNA binding protein in a biological sample according to the method of any of paragraphs 1 to 14; and
b) determining the positioning of one or more of the interactions in pre-RNA.

23. The method according to paragraph 22, wherein the positioning of one or more interactions is determined at an exon-intron boundary of alternative exons and/or flanking constitute exons and/or constitute exons.

24. The method according to paragraph 23, wherein an exon-intron boundary of alternative exons and/or flanking constitute exons and/or constitute exons is identified using an array.

25. A method for identifying an agent that modulates binding or association between an RNA an RNA binding protein of interest, comprising the steps of:

(a) determining an interaction between an RNA and an RNA binding protein in a biological sample according to the method of any of paragraphs 1 to 14 in the presence and absence of the agent; and
(b) determining if the agent modulates the binding or association between the RNA and the RNA binding protein of interest,
wherein a difference in the binding or association between the RNA and the RNA binding protein of interest in the presence of the agent is indicative that said agent modulates the binding or association.

26. The method of paragraph 25, wherein said method comprises the steps of:

(a) assessing a first level of binding or association between the RNA and the RNA binding protein in a first cell, wherein said first cell has been contacted with the agent;
(b) assessing a second level of binding or association between the RNA and the RNA binding protein in a second cell, wherein said second cell has not been contacted with the agent; and
(c) comparing said first level of binding or association with said second level of binding or association,
wherein a difference between said first level of binding or association and said second level of binding or association indicates an ability of said agent to modulate the association or binding between said RNA binding protein and said RNA.

27. A method for identifying an agent that modulates binding or association between an RNA an RNA binding protein of interest, comprising the steps of:

(a) preparing a map according to any of paragraphs 16 to 24 in the presence and absence of the agent; and
(b) determining if the agent modulates the binding or association between the RNA and the RNA binding protein of interest,
wherein a difference in the map obtained in the presence of the agent as compared to the map obtained in the absence of the agent is indicative that said agent modulates the binding or association.

28. A method for identifying an agent that modulates splicing regulation, comprising the steps of:

(a) preparing a map according to any of paragraphs 16 to 24 in the presence and absence of the agent; and
(b) determining if the agent modulates splicing regulation,
wherein a difference in the map obtained in the presence of the agent as compared to the map obtained in the absence of the agent is indicative that said agent modulates splicing regulation.

29. A nucleotide sequence comprising, consisting or consistent essentially of SEQ ID Nos. 1 to to 13 or a homologue, variant or fragment thereof.

30. A vector or a host cell comprising one or more of the nucleotide sequences according to paragraph 29.

31. A kit comprising a ligase, a protease, a reverse transcriptase, a first adapter, a reverse transcription primer, and optionally amplification primers and amplification reagents.

### Examples

### Materials & Methods

iCLIP analyses. HeLa cells were irradiated with UV-C light to covalently cross-link proteins to nucleic acids in vivo. Upon cell lysis, RNA was partially fragmented using low concentrations of RNase I, and hnRNP C-RNA complexes were immuno-purified with the antibody immobilized on immunoglobulin G-coated magnetic beads. After stringent washing, RNAs were ligated at their 3′ ends to an RNA adapter and radioactively labelled to allow visualization. Denaturing gel electrophoresis and transfer to a nitrocellulose membrane removed RNAs that were not covalently linked to the protein. Two size fractions of the RNA (FIG. 7a) were recovered from the membrane by proteinase K digestion. The oligonucleotides for reverse transcription contained two inversely oriented adapter regions separated by a BamHI restriction site as well as a barcode region at their 5′ end containing a two nucleotide barcode to mark the experiment and a three nucleotide random barcode to mark individual cDNA molecules. cDNA molecules were size-purified using denaturing gel electrophoresis, circularized by single-stranded DNA ligase, annealed to an oligonucleotide complementary to the restriction site and cut between the two adapter regions by BamHI. Linearized cDNAs were then PCR-amplified using primers complementary to the adapter regions (FIG. 7b) and subjected to high-throughput sequencing using Illumina GA2.

HeLa cells grown in a 10 cm plate were covered with ice-cold PBS buffer and subjected to UV-C irradiation (100 mJ/cm2, Stratalinker 2400). Upon removal of PBS buffer, cells were scraped off and transferred into microtubes (2 ml each). Cells were precipitated by centrifugation for 1 min at 14,000 rpm and shock frozen on dry ice.

For magnetic bead preparation, 50 μl of protein A-coated Dynabeads (Invitrogen) were washed 2× with 900 μl lysis buffer (50 mM Tris-HCl pH 7.4, 100 mM NaCl, 1 mM MgCl2, 0.1 mM CaCl2, 1% NP-40, 0.1% SDS, 0.5% Na-Deoxycholate). Dynabeads were resuspended in 200 μl lysis buffer, and 10 μg of hnRNP C antibody (Santa Cruz H-105) were added. After rotation at room temperature for 30-60 min, Dynabeads were washed 2× with lysis buffer and kept in last wash until addition of cross-linked lysate. Pellets were resuspended in 1 ml lysis buffer and sonicated.

For partial RNase digestion, RNase I (Ambion) was diluted 1:50 and 1:100 in lysis buffer for high and low RNase treatment, respectively. 10 μl RNase I dilution and 5 μl Turbo DNase (Ambion) were added to the cross-linked lysate and incubated for 3 min at 37° C. and 800 rpm. Cells were precipitated by two rounds of centrifugation at 4° C. and 14,000 rpm for 10 min followed by careful collection of the supernatant. The supernatant was added to Dynabeads and incubated for 1 h or overnight at 4° C. and 800 rpm. Dynabeads were washed 2× with high-salt wash buffer (50 mM Tris-HCl pH 7.4, 1 M NaCl, 1 mM EDTA, 0.1% SDS, 0.5% Na Deoxycholate, 1% NP-40) and 1× with PNK wash buffer (20 mM Tris-HCl pH 7.4, 10 mM MgCl2, 0.2% Tween-20).

For dephosphorylation of 3′ ends, Dynabeads were resuspended in 2 μl 10× Shrimp alkaline phosphatase buffer (Promega), 17.5 μl H2O and 0.1 μl Shrimp alkaline phosphatase (Promega) and incubated at 37° C. for 10 min with intermittent shaking (10 sec at 700 rpm followed by 20 sec pause). Samples were washed 2× with high-salt wash buffer, 1× with 900 μl PNK wash buffer and 1× with 50 μl 1×RNA ligase buffer (NEB, freshly prepared from frozen stock). For RNA linker ligation, 2 Dynabeads were resuspended in 15 μl L3 ligation mix (5 μl L3 RNA linker [5′-phosphate-UGAGAUCGGAAGAGCGGTTCAG-3′-Puromycin, 20 μM], 1.5 μl 10×RNA ligase buffer, 7.75 μl H2O, 0.5 μl RNasin [Promega], 0.25 μl RNA ligase [NEB]) and incubated overnight at 16° C. Samples were mixed with 5 μl NuPAGE loading buffer (Invitrogen), incubated for 5 min at 70° C. and placed on a magnetic stand to collect the eluate.

Samples were run on 9-well or 10-well Novex NuPAGE 4-12% Bis-Tris gels (Invitrogen) with 1×MOPS running buffer (Invitrogen). After gel electrophoresis, protein and covalently bound RNAs were transferred to a nitrocellulose membrane (Whatman) using a Novex wet transfer apparatus (Invitrogen). The nitrocellulose membrane was rinsed with 1×PBS, wrapped into cling film and exposed to a BioMax XAR Film (Kodak) at −80° C.

For isolation of cross-linked RNAs, 2 mg/ml proteinase K (Roche) was pre-incubated in PK buffer (100 mM Tris-HCl pH 7.5, 50 mM NaCl, 10 mM EDTA) for 5 min at 37° C. In order to recover different size fractions of RNAs, two fragments were cut out of the nitrocellulose membrane at different heights above the molecular weight of the protein (40 kDa). 200 μl proteinase K solution was added to each fragment and incubated for 30 min at 55° C. Incubation was repeated after addition of 130 μl PK/7 M urea buffer (100 mM Tris-HCl pH 7.5, 50 mM NaCl, 10 mM EDTA, 7 M urea). Samples were cooled to 37° C., mixed with 170 μl H2O and 600 μl RNA phenol/CHCl3 (Ambion) and incubated for 5 min at 37° C. and 1,100 rpm. After centrifugation for 10 min at 13,000 rpm and room temperature, 450 μl of the aqueous phase were transferred into a new microtube and again subjected to centrifugation. 400 μl of supernatant were mixed with 0.5 μl Glycoblue (Ambion), 40 μl 3 M sodium acetate pH 5.5 and 1 ml 100% EtOH and incubated overnight at −20° C. RNAs were precipitated by centrifugation for 30 min at 15,000 rpm and 4° C., washed with 500 μl 80% EtOH and resuspended in 12 μl H2O.

For reverse transcription, 1 μl RT primer (2 pmol/μl; the following three primers were used for replicates 1 to 3: 5′-phosphate-NNNCAAGATCGGAAGAGCGTCGTGGATCCT GAACCGCTC-3′; 5′-phosphate-NNNGAAGATCGGAAGAGCGTCGTGGATCCTGAACCGC-3′; 5′-phosphate-NNNTGAGATCGGAAGAGCGTCGTGGATCCTGAACCGCTC-3′; NNN represents 3-nt random barcode and bold nucleotides mark 2-nt barcode used as an experiment identifier) and 1 ml 10 mM dNTP mix were added to the RNA, preheated for 5 min to 70° C. and then held at 42° C. Once 6 μl RT mix (5 μl 5×RT buffer [Invitrogen], 1 μl 0.1 M DTT, 0.5 μl Superscript III reverse transcriptase [Invitrogen], 0.5 μl RNasin) were added and mixed by pipetting, reverse transcription was performed with the following program: 10 min at 42° C., 40 min at 50° C., 20 min at 55° C., and hold at 4° C. To remove RNA, samples were heated for 2 min to 95° C., mixed with 1 μl RNase A (Ambion) and incubated for 20 min at 37° C. cDNAs were precipitated by addition of 80 μl TE buffer, 0.5 μl Glycoblue, 10 μl 3 M sodium acetate pH 5.3 and 250 μl 100% EtOH, incubation for 1 h on dry ice or overnight at −20° C., and centrifugation for 30 min at 4° C. and 15,000 rpm. Pellets were washed with 500 μl 80% EtOH, dried for 3 min at room temperature and resuspended in 6 μl H2O.

For size separation, cDNAs were mixed with 2 μl 2×TBE-urea loading buffer (Invitrogen) and incubated for 3 min at 70° C. Samples were run on a 6% TBE urea gel (Invitrogen) in 1×TBE buffer for 40 min at 180 V. In order to recover different size fractions, two bands were cut from the gel corresponding to a cDNA size of 100-175 nt and 175-350 nt. Gel fragments were mixed with 400 ml TE buffer, crushed with a 1 ml syringe plunger and incubated for 2 h at 37° C. and 1,100 rpm. A Costar SpinX column (Corning Incorporated) was prepared by addition of two 1 cm glass wool pre-filters (Whatman 1823-101) and centrifugation for 1 min at 13,000 rpm. After transfer of the supernatant to the column, 40 μl 3 M sodium acetate pH 5.5 and 0.5 μl glycogen were added. Columns were vortexed before adding 1 ml 100% EtOH and incubating overnight at −20° C. Columns were washed by addition of 500 μl 80% EtOH and centrifugation for 10 min at 15,000 rpm and 4° C. Pellets were dried for 3 min at room temperature and resuspended in 12 μl H2O.

In order to circularize the cDNAs, samples were mixed with 1.5 μl 10× CircLigase buffer II (Epicentre), 0.75 μl 50 mM MnCl2 and 0.75 μl CircLigase II (Epicentre) and incubated for 1 h at 60° C. For subsequent linearization, a primer (5′-GTTCAGGATCCA CGACGCTCTTCAAAA-3′) complementary to the BamHI restriction site in the RT primer was annealed by adding 26 μl H2O, 5 μl FastDigest buffer (Fermentas) and 1 μl 10 μM primer and incubation with the following program: 2 min at 95° C., 70 cycles starting for 1 min at 95° C. and reducing the temperature with every cycle by 1° C. BamHI cleavage was performed by adding 3 μl Fastdigest BamHI (Fermentas) and incubating for 30 min at 37° C. Samples were mixed with 50 μl TE buffer, 0.5 μl Glycoblue, 10 μl 3 M sodium acetate pH 5.5 and 250 μl 100% EtOH and incubated for 1 h on dry ice or overnight at −20° C. cDNAs were precipitated by centrifugation for 30 min at 15,000 rpm and 4° C., washed with 500 μl 80% EtOH, dried for 3 min at room temperature and resuspended in 9 μl H2O.

For high-throughput sequencing, cDNAs were PCR-amplified by adding 0.3 μl Illumina paired-end primer mix (10 μM each; 5′-CAAGCAGAAGACGGCATACGAGAT CGGTCTCGGCATTCCTGCTGAACCGCTCTTCCGATCT-3′; 5′-AATGATACGGCGACCA CCGAGATCTACACTCTTTCCCTACACGACGCTCTTCCGATCT-3′, oligonucleotide sequences © 2006 and 2008 Illumina, Inc.) and 10 μl 2× Immomix (Bioline) and incubation with the following program: 10 min at 95° C., 35 cycles of [10 sec at 95° C., 10 sec at 65° C., 20 sec at 72° C.], 3 min at 72° C. In order to desalt the PCR products, a Microspin G-25 column (GE Healthcare) was resuspended by vortexing and spinned for 1 min at 735×g. Upon sample application, PCR products were re-eluted by centrifugation for 2 min at 735×g, and sequenced on an Illumina GA2 flow cell.

High-throughout sequencing and mapping. High-throughput sequencing of iCLIP cDNA libraries from three replicate experiments was performed on one lane of an Illumina GA2 flow cell with 54 nt run length. Sequence reads included a 2-nt barcode as unique experiment identifier plus a 3-nt random barcode that were introduced during cDNA synthesis. The obtained 6,544,506 sequence reads were separated per experiment based on the 2-nt barcode. In order minimize misassignments due to sequencing errors in the 2-nt barcode, cDNAs from different replicates starting at the same cross-link nucleotide and having the same 3-nt random barcode sequence were assigned to the replicate with the higher occurrence of this random barcode. Thus, replicates were actually separated based on 5-nt information at individual positions. The three expected 2-nt barcodes together represented 96% of all sequences (TG, 2,610,554; TC, 2,292,169; CA, 1,376,258; total, 6,278,981). The three no-antibody control samples were sequenced on Illumina GA2 flow cells with 54 nt run length (replicates 2 and 3 were sequenced together in one lane). The respective 2-nt or 3-nt barcodes as experiment identifiers were CA, ACT and AAG and identified 91,310, 122,957 and 71,044 reads, respectively (out of 5,782,612, 12,597,621 and 12,597,621 reads that were generated in total on the respective lanes).

Before mapping to the human genome, adapter sequences were removed from both ends of the sequence reads. In all hnRNP C and no antibody control experiments, the majority of sequences did not contain 3′ adapter sequences (hnRNP C: replicate 1, 74%; replicate 2, 75%; replicate 3, 84%; control: replicate 1, 91%; replicate 2, 37%; replicate 3, 75%), indicating that the respective inserts were longer than 49 nt.

Mapping of sequence reads was performed against the human genome (version Hg18/NCBI36) using bowtie version 0.10.11. After allowing one mismatch and 10 multiple hits (bowtie parameters-v 1-m 10-a), single hits were extracted by post processing.

Genomic annotations were assigned based on gene annotations given by UCSC (hg18.knownGene; 29,413 genes; FIG. 9).

High-throughout sequencing and mapping. High-throughput sequencing of iCLIP cDNA libraries from three replicate experiments was performed on one lane of an Illumina GA2 flow cell with 54 nt run length. Mapping of sequence reads was performed against the human genome (version Hg18/NCBI36) using bowtie version 0.10.1³¹. The 3-nt random barcode enabled us to discriminate PCR duplicates from sequences, which start at the same nucleotide, but derived from individual cDNA molecules. Random barcodes with more than one identical nucleotide were considered to be PCR duplicates, which were excluded from the data set (for more details, see Rot et al., manuscript in preparation). Following this strategy, a total of 3521462 sequences were removed from the analysis (85% of mapped reads), resulting in a final set of 309489, 216295 and 115566 sequences representing individual cDNA molecules from the three replicates. Last, the first nucleotide in the genome upstream of a mapping cDNA sequence was defined as ‘cross-link nucleotide’ and the total of corresponding cDNA sequences assigned as ‘cDNA count’ at this position. For subsequent analyses, replicates were merged into one iCLIP dataset by summing cDNA counts from all three replicates for each cross-link nucleotide.

The obtained 6,544,506 sequence reads were separated per experiment based on the 2-nt barcode. In order minimize misassignments due to sequencing errors in the 2-nt barcode, cDNAs from different replicates starting at the same cross-link nucleotide and having the same 3-nt random barcode sequence were assigned to the replicate with the higher occurrence of this random barcode. Thus, replicates were actually separated based on 5-nt information at individual positions. The three expected 2-nt barcodes together represented 96% of all sequences (TG, 2,610,554; TC, 2,292,169; CA, 1,376,258; total, 6,278,981). The three no-antibody control samples were sequenced on Illumina GA2 flow cells with 54 nt run length (replicates 2 and 3 were sequenced together in one lane). The respective 2-nt or 3-nt barcodes as experiment identifiers were CA, ACT and AAG and identified 91,310, 122,957 and 71,044 reads, respectively (out of 5,782,612, 12,597,621 and 12,597,621 reads that were generated in total on the respective lanes).

Before mapping to the human genome, adapter sequences were removed from both ends of the sequence reads. In all hnRNP C and no antibody control experiments, the majority of sequences did not contain 3′ adapter sequences (hnRNP C: replicate 1, 74%; replicate 2, 75%; replicate 3, 84%; control: replicate 1, 91%; replicate 2, 37%; replicate 3, 75%), indicating that the respective inserts were longer than 49 nt.

Mapping of sequence reads was performed against the human genome (version Hg18/NCBI36) using bowtie version 0.10.11. After allowing one mismatch and 10 multiple hits (bowtie parameters-v 1-m 10-a), single hits were extracted by postprocessing.

Genomic annotations were assigned based on gene annotations given by UCSC (hg18.knownGene; 29,413 genes; FIG. 9).

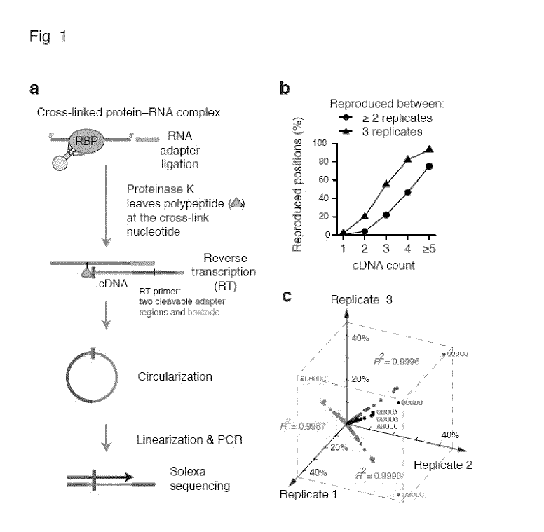

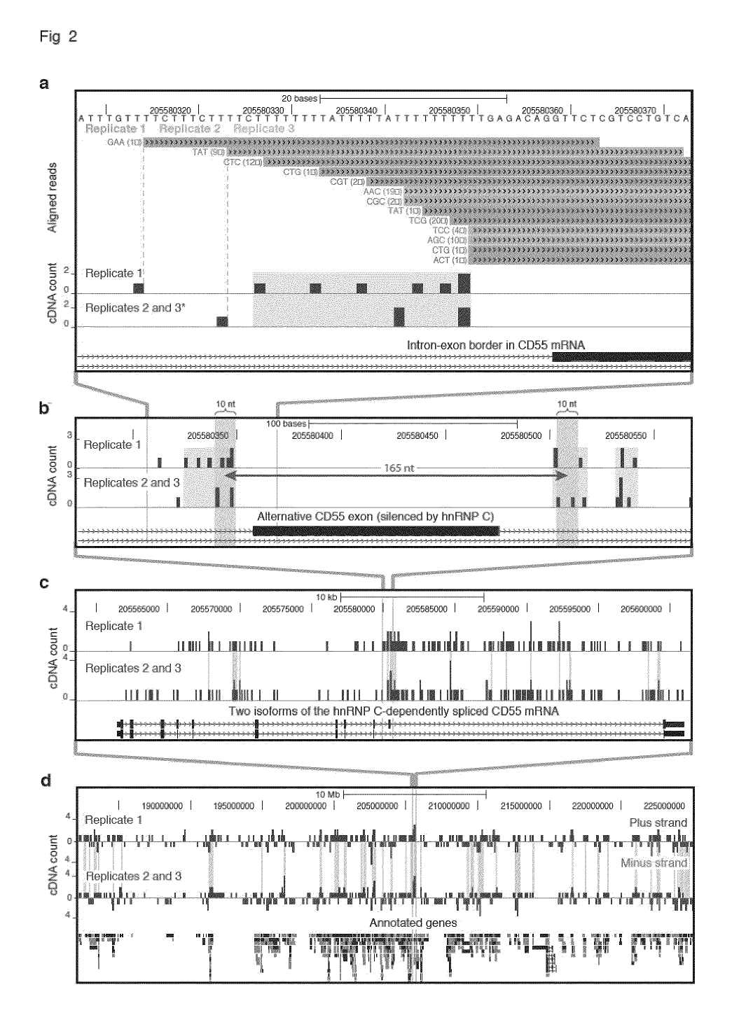

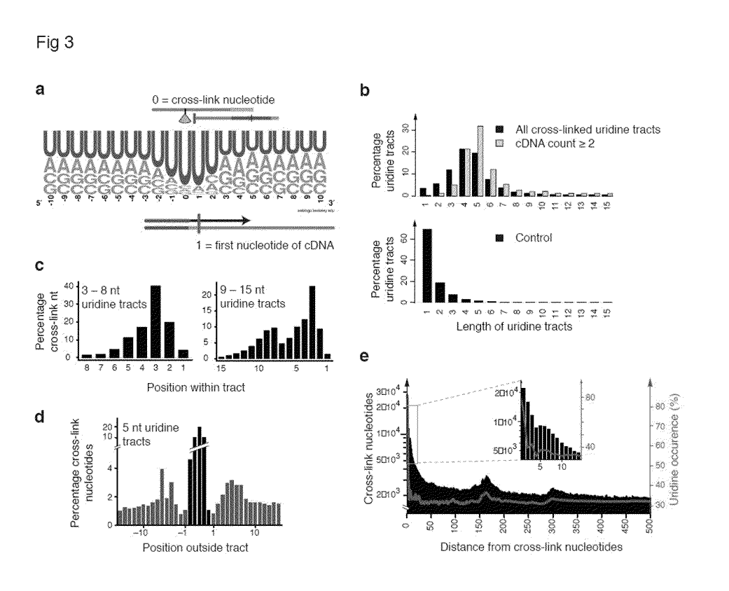

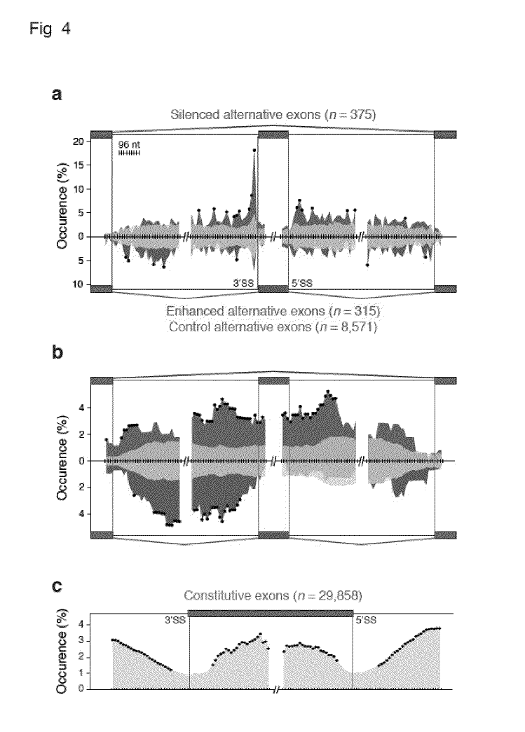

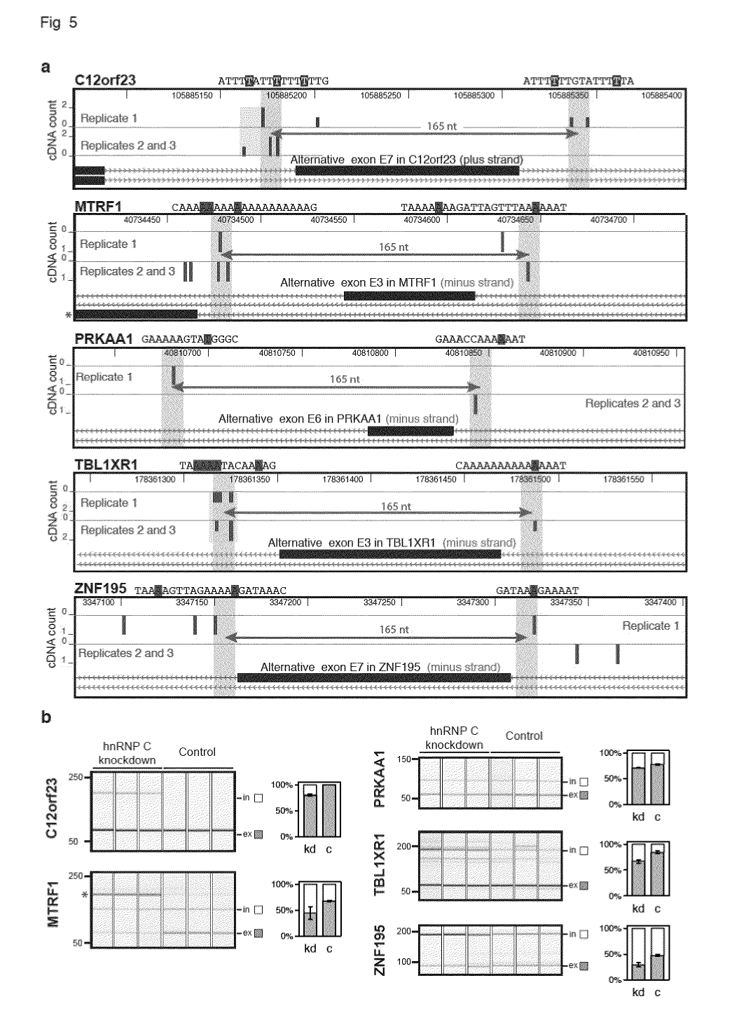

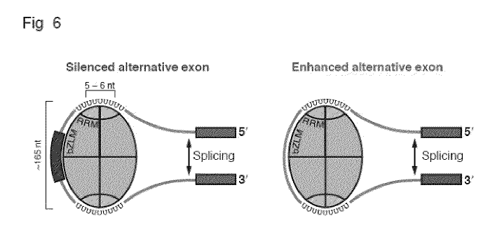

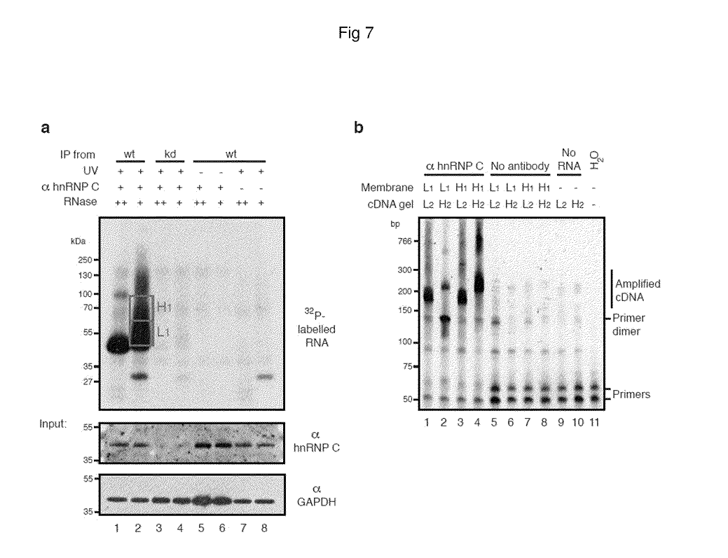

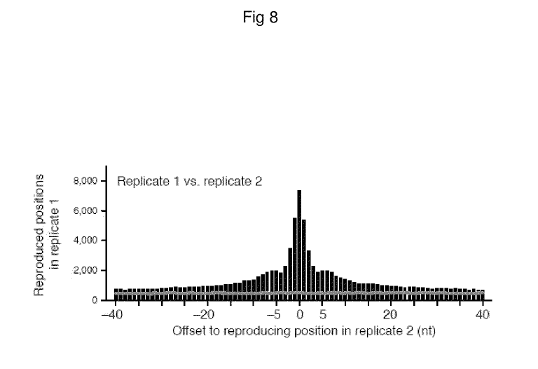

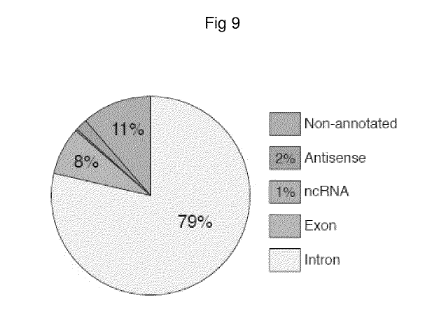

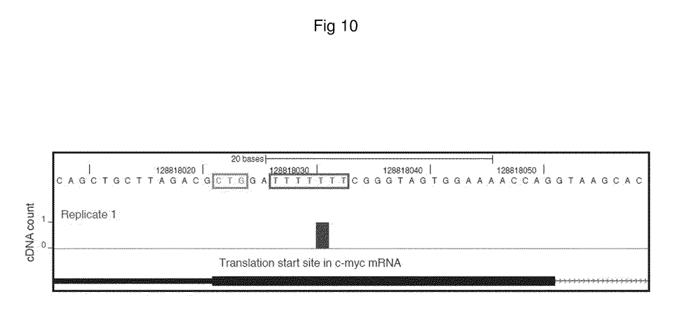

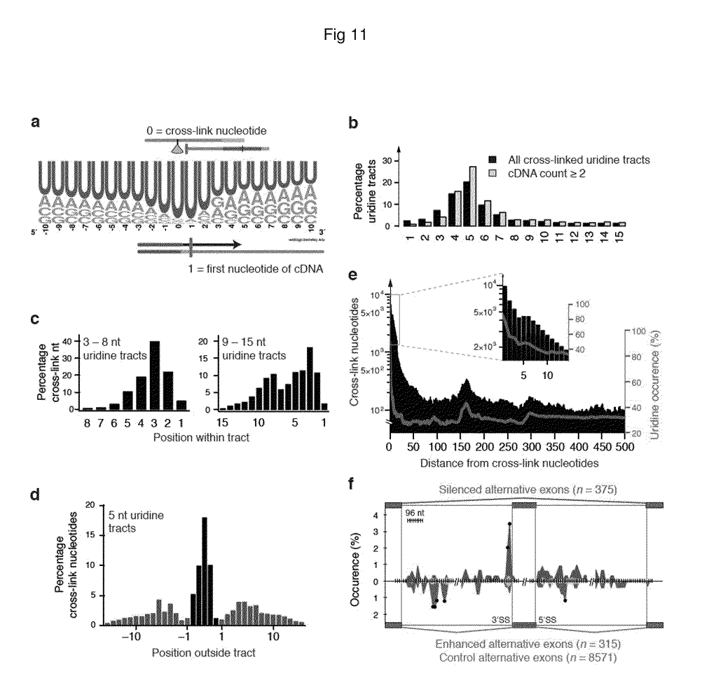

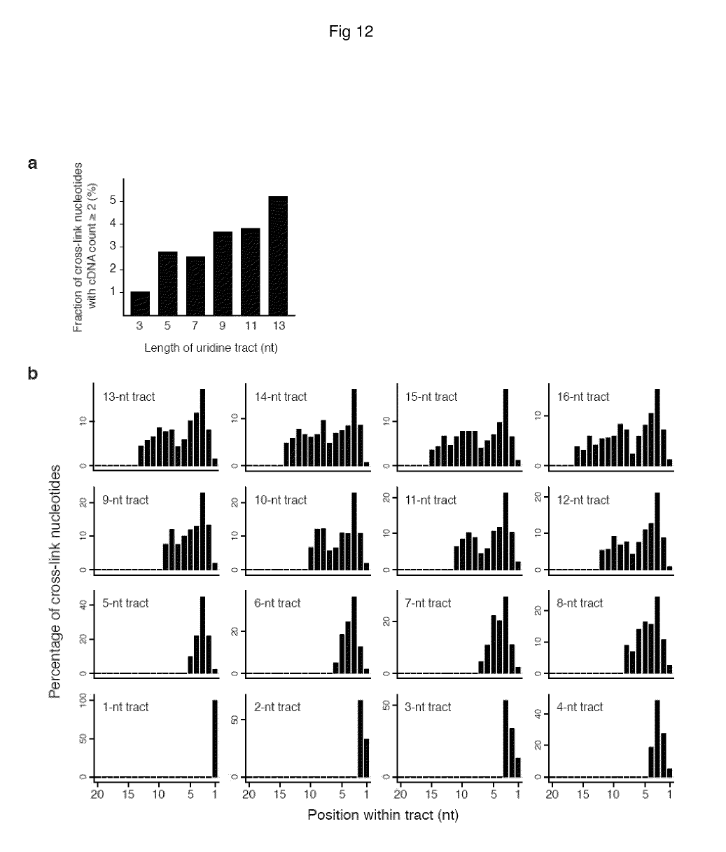

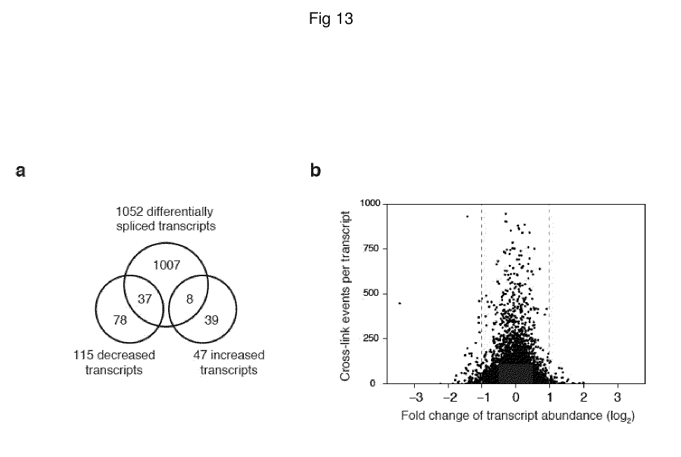

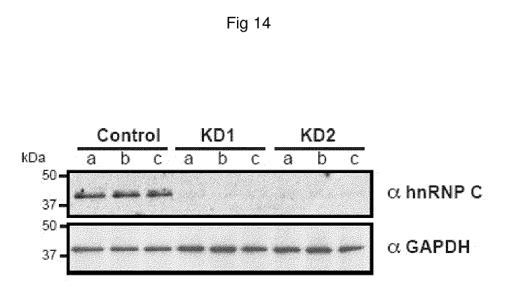

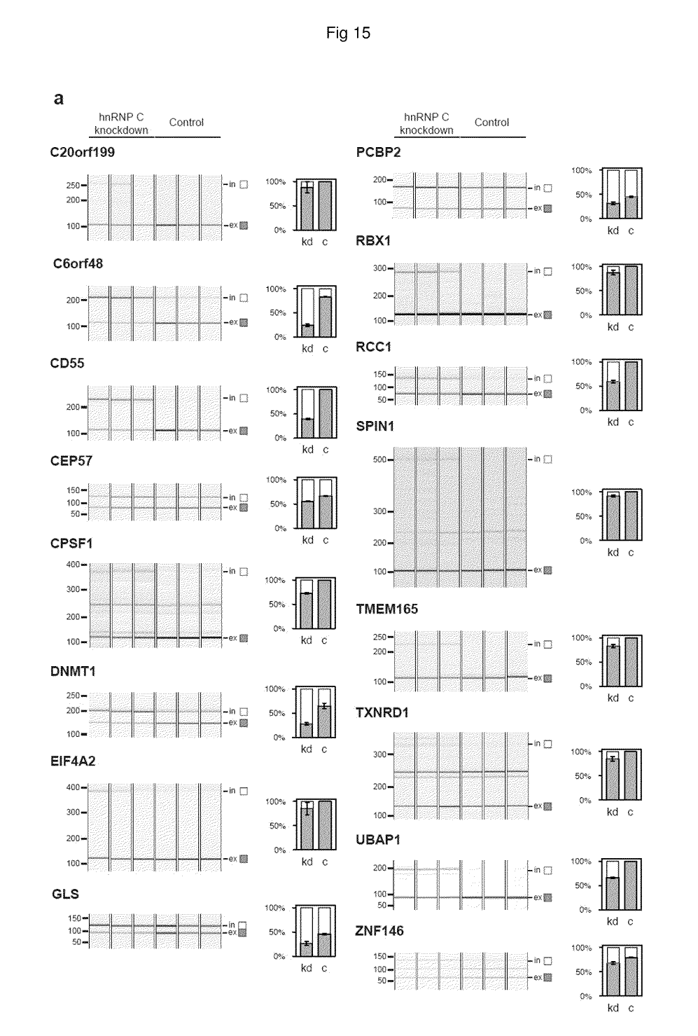

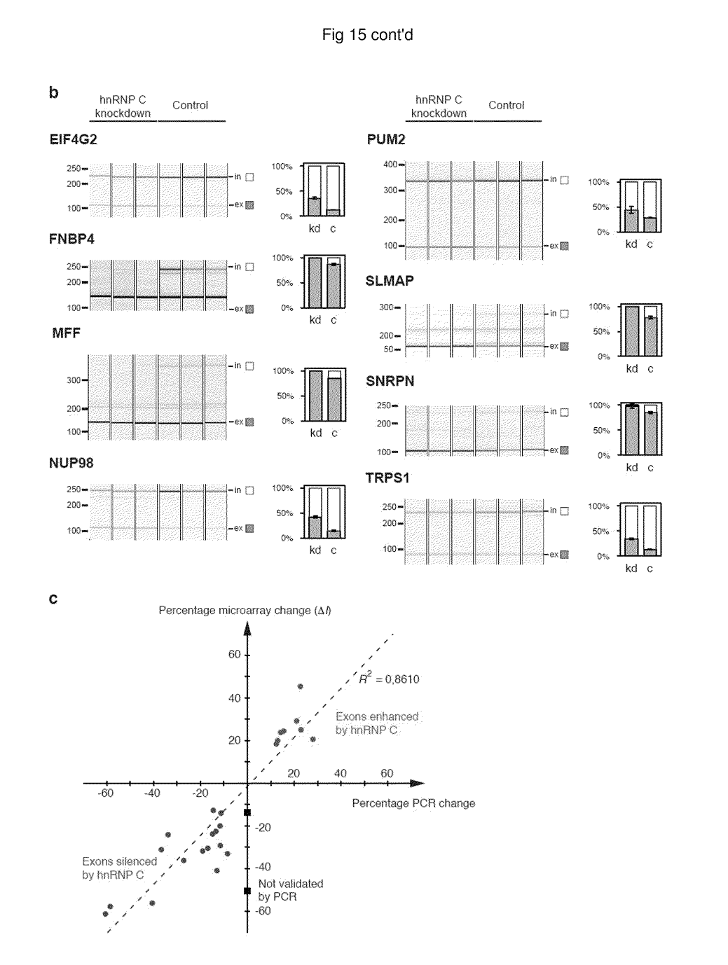

Reproducibility analyses. Reproducibility of cross-link nucleotides at single nucleotide resolution (FIG. 1b) was determined by counting the number of cross-link nucleotides with a given cDNA count that were present in two or three replicates. If reproducing cross-link nucleotides harbored identical cDNA count values, all except one were excluded from the count. Thereby, the resulting total number of cross-link nucleotides with a given cDNA count reflects equal the total number of positions in the genome that were identified with that cDNA count. The number of cross-link nucleotides of a given cDNA count that were reproduced in at least two or all three replicates is given as a fraction of the total number of cross-link nucleotides with that cDNA count identified within the genome.

In order to determine the offset of reproducing positions (FIG. 8), cross-link nucleotides of hnRNP C iCLIP replicate 1 were compared against replicate. For each cross-link nucleotide in replicate 1, we summarized the offset of all surrounding cross-link nucleotides in replicate 2 up to a distance of 40 nt. Positive or negative offset values indicate whether the reproducing position in replicate 2 locates downstream or upstream of the cross-link nucleotide in replicate 1, respectively. In order to assess the expected background distribution, replicate 1 was also compared against a randomized version of replicate 2. Randomization was performed as described above. The false-discovery rate (FDR) for each position was determined according to Yeo and coworkers¹².

Analysis of sequence and positioning of cross-link nucleotides. All analyses of hnRNP C binding were based on sequences mapping to human nuclear chromosomes. In order to determine pentanucleotide frequencies at cross-link nucleotides (FIG. 1c), we assessed all pentanucleotides overlapping each cross-link nucleotide within the three replicate experiments. Multiple occurrences at the same cross-link nucleotide were counted only once. Frequencies were calculated as the number of cross-link nucleotides that are associated with a certain pentanucleotide.

For calculating base frequencies of iCLIP sequence reads (FIG. 3a), we extracted genomic sequence corresponding to the first 10 nucleotides of all reads plus 11 nucleotides of preceding sequence. Graphic representation was generated using Weblogo 3³² (http://weblogo.berkeley.edu). Background distribution of bases was calculated using all transcribed regions annotated in the Ensembl database³³ (release 54; http://www.ensembl.org/). In order to determine the lengths distribution of uridine tract bound by hnRNP C (FIG. 3b), we extracted all uridine tracts in the genome that harbored at least one cross-link nucleotide. Distribution of uridine tracts within the transcriptome was calculated again based on all transcribed regions.

The percentage of cross-link nucleotides located within a tract of at least four uridines was calculated as a fraction of all identified cross-link nucleotides. The expected background was calculated upon randomization of cross-link nucleotide positions as described above. Finally, the expected value for background localization to tracts of at least four uridines was calculated as mean percentage from 100 random permutations.

In order to assess the spacing of cross-link nucleotides (FIG. 3e), we summarized the distances of all cross-link nucleotides to all downstream cross-link nucleotides within a window of 500 nt. In order to analyze the short-range binding patterns, we summarized all cross-link nucleotides on each position of uridine tracts of the same length (FIG. 3c, FIG. 12b). For tracts of five uridines, we additionally assessed distribution of surrounding cross-link nucleotides (FIG. 3d), using only those tracts that displayed at least one additional cross-link nucleotide at a distance of no more than 15 nt to either side.

In order to examine the influence of uridine tract length on the occurrence of cross-linking (FIG. 12a), the percentage of tracts with a cDNA count of at least two at the third position from the 3′ end was calculated relative to all tracts of the same length containing a cross-link site at this position.

Knockdown of hnRNP C. hnRNP C was depleted in HeLa cells using two different siRNAs.

Splice-junction microarrays. Microarray analyses and PCR validations were performed as described herein. The microarray data was analyzed using ASPIRE version 3 that was modified relative to previous versions^{11,34} by adding background subtraction and significance ranking of predicted splicing changes. By analysing the signal of reciprocal probe sets, ASPIRE3 was able to monitor 53632 alternative splicing events. Applying a threshold of |Dirank|≧1, we identified 1340 differentially spliced alternative exons, of which 662 and 678 were increased and decreased in the hnRNP C knockdown cells, respectively.

RNA map. In order to analyze the impact of hnRNP C positioning on splicing regulation, we assessed the positioning of hnRNP C cross-link sites at exon-intron boundaries of alternative exons and flanking constitutive exons (as annotated for the Affymetrix microarray), including 45 nt of exonic and 315 nt of intronic sequence (FIG. 4a, b). In addition, 348 nt of exonic and 372 nt of intronic sequence were analyzed at the exon-intron boundaries of constitutive exons (FIG. 4c). When introns or exons were shorter than two times the length of the analyzed area, analysis was restricted up to the middle of this intron or exon, respectively. For all RNA maps, regions were divided into non-overlapping windows of 12 nucleotides. For each window, the number of cross-link nucleotides was counted as 1 if at least one crosslink nucleotide resided within this window. Thus, the resulting occurrence value reflects the number of exons with at least one cross-link nucleotide within this window. When positioning of particles was analyzed (FIG. 4b, c), only cross-link nucleotides with a spacing of 160-170 nt as well as all intervening nucleotides were taken into account. For all RNA maps, percentages were calculated by dividing the number of exons that have at least one cross-link nucleotide within a given window by the total number of exons analyzed at this window.

Randomization of iCLIP cross-link nucleotide positions. As a control for bioinformatic analyses, iCLIP cross-link nucleotide positions were randomized as follows: In order to account for potential differences in transcript abundance, crosslink nucleotides were assigned to transcript regions that are expected to have a common expression level. To this end, exons were separated from introns, non-coding RNA genes within introns from the rest of intronic regions, and untranslated regions from coding sequence based on gene annotations given by UCSC (hg18/NCBI36).

Since exons are generally small, all exons of a given gene were concatenated into one region. Randomization was performed within these regions considering cDNA counts, such that e.g. for a position of cDNA count=2 within an intron, two positions were randomly selected within the same intron during randomization.

Evaluation of significance of hnRNP C cross-link nucleotides. In order to determine the false-discovery rate (FDR) for each position, we applied a strategy similar to the approach used by Yeo and coworkers2 performing the following steps:

(i) Cross-link nucleotides were assigned to transcript regions as described for randomization above. Both coding and non-coding genes were included (in case of overlapping genes, the cross-link nucleotide was assigned to the shorter gene). Crosslink nucleotides in antisense orientation to the associated gene or locating to nonannotated genomic regions were removed.

(ii) Cross-link nucleotides were extended by 15 nt to both directions. Subsequently, we calculated the height at each cross-link nucleotide as the total number of overlapping extended cross-link nucleotides at this position by adding up their cDNA counts.

(iii) The distribution of heights was defined as follows: The height h at a cross-link nucleotide position lies within the interval [1,H], where H is the maximum observed height within a given region. nh and N donate the number of cross-link nucleotides with height h and of total cross-link nucleotides within the same region, respectively. The resulting distribution of heights is {n1, n2, . . . nh, . . . nH−1, nH}. Thus, the probability of observing a height of at least h is Ph=Σ ni(i=h, . . . , H)/N. (iv) The background frequency was computed by 100 iterations of randomization as described above. The modified FDR for a cross-link nucleotide with height h was computed as FDR(h)=(μh+σh)/Ph, where ph and ah are the average and standard deviation, respectively, of Ph, random across the 100 iterations. This identified 33,991 cross-link nucleotides as part of significant hnRNP C binding clusters which were referred to as clustered cross-link nucleotides (FDR<0.05).

Knockdown of hnRNP C. In order to knockdown hnRNP C in HeLa cells, we independently used two different HNRNPC Stealth Select RNAi™ siRNAs (KD1 and KD2 refer to siRNAs HSS179304 and HSS179305 from Invitrogen, respectively) at a final concentration of 5 nM. The siRNAs were transfected using Lipofectamine™ RNAiMAX (Invitrogen) according to the manufacturer's instructions (protocol for forward transfection). Control samples were generated using Stealth RNAi™ siRNA Negative Control (Invitrogen) following the same procedure. Knockdown efficiency was controlled by Western blot analyses using hnRNP C-specific antibodies (FIG. 14). For microarray analysis, KD1a, KD1b and KD2a were used whereas for RT-PCR analyses KD1c, KD2b and KD2c were used.

Splice-junction microarrays. mRNA from hnRNP C knockdown and control HeLa cells was purified using the RNeasy MinElute Cleanup Kit (Qiagen) combined with the RiboMinus™ Eukaryotic Kit for RNAseq (Invitrogen). Labeled sense cDNA for microarray hybridization was prepared using GeneChip® WT Sense Target Labeling and Control Reagents (Affymetrix) according to the manufacturer's instructions, but replacing the included Superscript II with Superscript III (Invitrogen). Labeled samples were hybridized to the non-commercial human exon-junction microarray (HJAY, Affymetrix).

PCR validations. In order to validate the splicing changes identified in our splicejunction microarray analyses, we performed quantitative PCR measurements (Tables 4, 5; FIG. 5b; FIG. 16) using BIOTaq polymerase (Bioline) under the following conditions: 95° C. for 5 minutes, 40 cycles of [95° C. for 15 seconds, 60° C. for 15 seconds, 72° C. for 30 seconds], then finally 72° C. for 3 minutes. A QIAxcel capillary gel electrophoresis system was used to visualize the PCR products. A photomultiplier detector converted the emission signal into a gel image and an electropherogram that allowed visualization and quantification of each PCR product, respectively. All measurements were performed in three replicates.

ASPIRE3 algorithm. The high-resolution splice-junction microarray was produced by Affymetrix, monitoring 260,488 exon-exon junctions (each with 8 probes) and 315,137 exons (each with 10 probes). cDNA samples were prepared using the GeneChip WT cDNA Synthesis and Amplification Kit (Affymetrix). Analysis of microarray data was done using version 3 of ASPIRE (Analysis of SPlicing Isoform REciprocity). ASPIRE3 predicts splicing changes from reciprocal sets of microarray probes that recognize either inclusion or skipping of an alternative exon. The primary difference in version 3 of ASPIRE software relative to the previous versions is that background detection levels are experimentally determined for each probe, allowing to subtract the background in a probe-specific manner. By analysing the signal of reciprocal probe sets, ASPIRE3 was able to monitor 53,632 alternative splicing events.

The following nomenclature is used:

TA—estimated absolute transcript abundance (arbitrary value)
ΔT—fold change in transcript abundance
ΔT rank—modified t-test to sort the genes based on ΔT significance
I—estimated percentage of exon inclusion
ΔI—estimated change in percentage of exon inclusion
ΔI rank—modified t-test to sort the exons based on ΔI significance

The analysis includes the following basic steps:

1. All probe sets were mapped to human transcripts (positional gene annotations given by Affymetrix) and linked to the x/y coordinates of the individual probes on the microarray. Detected exons were categorized as constitutive or alternative. For the former, probes were combined into reciprocal groups that detect exon inclusion (Ein) or exon skipping (Eex). Constitutive exons were only monitored by Ein probes.

2. For each probe, background percentiles were experimentally determined by hybridizing the microarray with labeled 33 nt and 34 nt random oligonucleotides. Background detection probes were grouped according to their GC content, and for each group the background signal percentiles (5%, 17.5%, 32.5%, 47.5%, 62.5%, 75%, 84%, 91%, and 97%) were calculated. Each probe on the microarray was then assigned to its specific group of background detection probes that shared the same GC content. This allows determination of background values for each probe based on a subset of background detection probes with equal GC content that should detect a similar background signal.

3. Data from CEL files were normalized by background values. To this end, replicate specific percentile values were calculated for each group of background detection probes and subtracted from the signal values of the respective probes with the same GC content. Resulting values<0 were set to 0. Finally, values for each experiment were normalized by total signal on the microarray to correct for inter-replicate variations. The resulting values represent the fold-enrichment of signal relative to background.

4. Upon removal of outliers with high variation, signal values were weighted according to their signal intensity and variation. To this end, the probe weight (NUM) was determined by first calculating value X as the quotient of average and standard deviation within each set of reference (1) and experimental (2) samples. X values>5 were set to 5. Value Y was then determined according to the higher of the two average values: if average<50, Y was set to average/50, if 50<average<1000, Y was set to 1, and if average>1000, Y was set to 1000/average. Finally, NUM was calculated as the product of Y and the average of both X values. Probes with NUM<1 were excluded from further analyses.

5. Abundance and change of each transcript cluster were assessed by collecting all probes from probe sets categorized as constitutive within each transcript cluster. If this gained less than 15 non-filtered probe values, also probe sets categorized as alternative were taken into account. For each replicate, weighted average values were calculated for each considered probe (VAL1 . . . VALx, where x stands for the number of considered probes in the transcript cluster) within each transcript cluster and integrated into a value of transcript cluster abundance (TA): TA=((VAL1×NUM1)+ . . . +(VALx×NUMx))/n, where n is the sum of all respective probe weights (NUM1 10+ . . . +NUMx). Then, the probe ratio (R) was determined for each probe as the quotient of median probe values for reference (1) and experimental (2) samples. Finally, the transcript cluster change (ΔT) was calculated as follows: M(log 2(R))=((log 2(R1)×NUM1)+ . . . +(log 2(Rx)×NUMx))/n, and ΔT=2M(log 2(R)).

6. Probe values were normalized relative to the transcript cluster change to account for gene-specific changes in transcription and RNA degradation, allowing to specifically analyze changes in alternative splicing. To this end, all probe values in reference or experimental samples were divided or multiplied, respectively, by the square root of ΔT for the corresponding transcript cluster. Based on the assumption that all probes within a probe set should detect the same transcript isoform and should thus have the same average signal, each probe set value was divided by its own average in all replicates and then multiplied by the average value of all probes within the given probe set over all replicates. This resulted in normalized probe values that were used in all subsequent steps (except for ranking the significance of transcript changes).

7. Exon abundance (A) and percentage of exon inclusion (I) were determined by first calculating a weighted average over all probes within a probe set for each replicate (VAL1 . . . VALx): A=((VAL1×NUM1)+ . . . +(VALx×NUMx))/n. For reciprocal sets of both Ein and Eex, the percentage of exon inclusion (I) was calculated as I=Aein×100/(AEin+AEex). For all Ein probes without reciprocal Eex probes, the replicate with the highest exon abundance was taken as 100% and I was calculated by dividing each exon abundance value by the respective value of this replicate. Finally, changes in exon inclusion (ΔI) were detected by evaluating the difference of the averages over all I values of the two sets of samples.

8. Reciprocal probe set pairs were re-analyzed to rank exons by the predicted splicing change (ΔI rank). To this end, the significance of the difference in average probe values within a probe set was assessed as follows: The weighted average of all probe values in the probe set was determined as AV=((VAL1×NUM1)+ . . . +(VALx×NUMx))/n, and S calculated as the square root of the sum of squared standard deviations of probes in sample sets 1 and 2. If 4×S was smaller than a quarter of the average of AV values of sample set 1 and 2, S was set to the latter value. Value Test was then calculated as the difference of individual averages of sample sets 1 and multiplied by the square root of N minus 1 and divided by S, where N stands for the number of probes with non-filtered values in the probe set (this should be 8 probes in an exon-exon border set and 10 in an exon probe set, if none of the probe values were filtered out). Finally, ΔI rank was calculated as ΔI×TestEin/400, if only Ein probes were available for the exon as it is the case for constitutive exons. When Ein and Eex probe sets detect the reciprocal signal change, their Test values will have opposite signs, therefore subtracting them will rank the exon higher in significance. If the absolute value of TestEin is smaller than the absolute value of TestEex, ΔI rank was calculated as ΔI×(2×TestEin−TestEex)/200, or as ΔI×(TestEin−2×TestEex)/200, if the opposite is true, since doubling the value of the probe set with the smaller Test value gives a stronger weight to the reciprocity of the change. Exons with ΔI rank>1 were predicted as enhanced or silenced in the experimental sample set.

9. In order to rank transcripts by the predicted transcript cluster change (ΔT rank), we first normalized all corresponding probe values to their average values over all replicates and within the complete set following the assumption that they detect the same transcript (normalized probe value=probe value×average value of all probes corresponding to this transcript cluster over all replicates/average value of the given probe over all replicates). Then, the two sets of probe values were compared (all probes and all replicates of one experiment within the same transcript cluster). To this end, AV and S values were calculated as described in 8. and integrated into ΔT rank=(log 2ΔT×((AVSample1−AVSample2)×√(N−1)/S)/20, where N is the number of probes with non-filtered values in the transcript cluster (most transcript clusters contain more than 100 probes).

iCLIP Maps hnRNP C Binding to Pre-mRNAs at Nucleotide Resolution

We employed iCLIP to examine the positioning of hnRNP C on pre-mRNAs in vivo. Three replicate iCLIP experiments were performed using an hnRNP C antibody on human HeLa cell lysates. The purified protein-RNA complex was absent when omitting UV-cross-linking or the use of hnRNP C antibody, and was diminished when hnRNP C knockdown cells were used (FIG. 7a). Cross-linked RNA was reverse transcribed and PCR amplified, controlling PCR specificity with an experiment that lacked the antibody during purification (FIG. 7b). High-throughput sequencing using Illumina GA2 generated a total of 6.5 million sequence reads (Table 1). 4.2 million sequence reads aligned to the human genome by allowing only single genomic hits and one nucleotide mismatch. Next, we eliminated PCR amplification artifacts by removing sequences that truncated at the same nucleotide in the genome and shared the same random barcode. This identified 641350 reads in total for the three replicate experiments, each representing a uniquely cross-linked RNA molecule. Finally, we summarized the number of sequences at each cross-link nucleotide into a ‘cDNA count’, representing a quantitative measure of the amount of hnRNP C cross-linking to each position (FIG. 2a). For the analyses of three independent no-antibody control samples we generated a total of 18 million sequence reads. After elimination of PCR amplification artifacts only 1780 unique cDNAs remained (Table 1), reflecting the high quality of purification and library preparation steps.

The iCLIP data were of high positional precision. Reproducibility of iCLIP data was demonstrated by the observation that 12790 cross-link nucleotides were identified in at least two independent experiments (FIG. 1b, 2a). 75% of cross-link nucleotides with a cDNA count of five or more were seen in all three experiments showing that the strongest cross-link sites of hnRNP C are the most reproducible (FIG. 1b). Furthermore, there was an enrichment of cross-link nucleotides with an offset of one or two nucleotides (FIG. 8). This observation may arise from protein contacts to more than one nucleotide of the RNA. In addition, the steric hindrance of the peptide fragment remaining on RNA may cause reverse transcription to terminate more than one nucleotide upstream of the cross-link site. As an independent measure of reproducibility we compared the occurrence of pentanucleotides overlapping the cross-link nucleotides; we found a high correlation between the three experiments (FIG. 1c), underlining the high precision of iCLIP in capturing protein-RNA interactions.

iCLIP identified large-scale binding of hnRNP C across the whole transcriptome. Although only a few direct targets were known prior to this study, we found hnRNP C cross-linking to transcripts from 55% of all annotated protein-coding genes (FIG. 9, FIG. 2). This places hnRNP C as a major post-transcriptional regulator of similar importance as, for example, the poly-pyrimidine tract-binding protein (PTB) that was shown to bind transcripts of 43% of annotated human genes¹⁴. Among previously described hnRNP C targets, we observed binding to the regulatory element that determines start codon selection within the c-myc mRNA and to the 3′ untranslated region of the APP mRNA^{15,16} (FIG. 10). 79% of cDNAs mapped in a sense orientation relative to introns, 9% to exons and 1% to non-coding RNAs. 11% mapped to intergenic regions, indicating that these harbor previously undescribed transcribed regions. Only 2% mapped in an antisense orientation relative to annotated genes, confirming that iCLIP generates strand-specific information on RNA binding (FIG. 9, FIG. 2d). In summary, our data demonstrate that hnRNP C has a central role as a regulator of nascent transcripts.

In order to reduce false positive hits and to increase the resolution of the data, previous CLIP studies have applied filtering algorithms to identify CLIP cDNA clusters in genome. Applying this approach to the hnRNP C dataset, we identified 33991 clustered cross-link nucleotides (FDR<0.05)¹². This filtering removed 94% of all cross-link nucleotides, which most likely included true binding sites. Since the iCLIP libraries prepared during this study are not fully saturated—a limitation that currently applies to all CLIP methods—many real binding sites are currently represented by only few cDNAs. This view was supported by the observation that 6367 out of 12790 reproduced cross-link nucleotides were removed during the filtering process. Therefore, we performed all the analyses described below on the complete and the filtered datasets; as shown in FIG. 11, the results are quantitatively and qualitatively similar, indicating that both sets are of high quality. Therefore in order to minimize loss of information, we describe findings for the complete dataset in the remainder of this work.

hnRNP C Cross-Links to Uridine Tracts

The high resolution of iCLIP data allowed us to assess the sequence specificity of hnRNP C binding. Strikingly, uridine represented 85% of cross-link nucleotides (p-value<0.001 by hypergeometric distribution for enrichment relative to background base frequencies; FIG. 3a). Surrounding positions were also strongly enriched for uridines, such that 65% of cross-link nucleotides were part of a contiguous tract of four or more uridines (FIG. 3b). These results agree with the in vitro observation that the RRM domains of hnRNP C bind to uridine tracts¹⁷⁻¹⁹, suggesting that cross-link nucleotides reflect the positions where the RRM domains contact RNA in vivo. In comparison, only 15-24% of cross-link nucleotides from the no-antibody control experiments were located in a tract of four or more uridines, demonstrating a significant enrichment of uridine tract binding in the hnRNP C iCLIP data (p value<0.01 by Student's t-test). We note that the control displays a bias to bind uridine tracts compared with the expected 5% from the background distribution in transcribed regions. However, this is in line with previous studies on single-stranded DNA-binding proteins that show preferential cross-linking to thymidine residues^{20,21}. Nonetheless, the small number of sequence reads and the low cross-linking bias in the control data contrast the strong preference for uridine by hnRNP C, indicating that the vast majority of iCLIP sequence reads reflect real hnRNP C binding events. Furthermore, the ability of iCLIP to quantify the number of cDNAs mapping to each cross-link nucleotide allowed us to analyze the affinity of hnRNP C to uridine tracts of different lengths. We found that cDNA counts increased with the number of uridines in the tract, suggesting that hnRNP C binds longer tracts with higher affinity (FIG. 3b, FIG. 11b, 12a).

The Spacing of Cross-Link Sites Reflects hnRNP Particle Formation

iCLIP allowed us to resolve adjacent binding sites within uridine tracts. We found that regardless of the length of the uridine tract, hnRNP C primarily cross-linked to the third uridine from the 3′ end (FIG. 3c, FIG. 11c, 6b). In addition, we identified a second peak of hnRNP C cross-linking positioned five or six nucleotides upstream on tracts longer than nine uridines. Consistently, such dual binding also occurred on shorter tracts when flanked by neighboring uridine tracts (FIG. 3d, FIG. 11d). Since the hnRNP C tetramer binds RNA with two RRM domains positioned proximally to each other^{6,22}, the dual cross-linking pattern could result from adjacent binding by the two RRM domains. These results show that the high resolution of iCLIP can elucidate combinatorial binding by multiple RNA-binding domains to proximal RNA binding sites, which would otherwise remain unresolved.

In addition to the short-range spacing within uridine tracts, iCLIP also identified a pattern of long-range spacing of cross-link nucleotides. We found peaks at distances of 165 and 300 nucleotides (FIG. 3e, FIG. 11e). Strikingly, the uridine density also peaked at the same positions (FIG. 3e, FIG. 11e). The defined spacing between cross-link nucleotides suggests that the intervening RNA is incorporated into the hnRNP particles. This model agrees with the organization of hnRNP particles as proposed by previous studies^{6,23,24}. Taken together, the precise mapping of hnRNP C cross-link sites provides insights into the structure of hnRNP particles.

The Positioning of hnRNP Particles Determines the Splicing Outcome

iCLIP allowed us to assess precisely the positioning of hnRNP C on alternatively spliced pre-mRNAs. Comparing transcript abundance from hnRNP C knockdown and control HeLa cells using high-resolution splice-junction microarrays, we detected significant increases and decreases by a factor of at least 2 for 47 and 115 transcripts, respectively (p-value<0.01 by Student's t-test). Transcript changes showed no apparent correlation with the amount of hnRNP C cross-linking (FIG. 13). By far the strongest change was seen for the hnRNP C transcript (decreased by a factor of 10), underlining the efficiency and specificity of the knockdown, which was also verified by Western blot analysis (FIG. 14). Using the ASPIRE3 algorithm, we detected changes in splicing at 1340 alternative exons. Transcripts harboring at least one alternatively spliced exon were significantly over represented among the differentially expressed transcripts and vice versa (FIG. 13b), indicating a relation between alternative splicing and transcript abundance. We observed a similar incidence of increased or decreased exon inclusion in hnRNP C knockdown cells, indicating that hnRNP C can either silence or enhance exon inclusion, respectively. We validated changes at 26 exons by RT-PCR with a 92% success rate (Table 2; FIG. 16). In order to address the role of hnRNP C binding in these changes, iCLIP data and splicing profiles were integrated into an ‘RNA map’²⁵. Increased density of cross-link nucleotides was seen at the splice sites of silenced alternative exons (FIG. 4a, FIG. 11f). At the 3′ splice site, hnRNP C predominantly cross-linked within the first 30 nucleotides that generally coincide with the poly-pyrimidine tracts, as seen in the CD55 pre-mRNA (FIG. 2a, FIG. 4a). This suggests that similar to PTB, hnRNP C can regulate alternative splicing by repressing specific 3′ splice sites²⁶. In conclusion, the ability of iCLIP to map cross-link nucleotides to characterized RNA regulatory elements can indicate the function of protein-RNA interactions.

In order to understand the impact of higher-order hnRNP particles on the observed splicing changes, we restricted the analysis to the cross-link sites displaying long-range spacing indicative of particle formation. We considered the regions between these cross-link sites as being incorporated into the particles. Due to the limited complexity of the clustered dataset, we restricted this analysis to the complete dataset. We found that silenced exons and proximal intronic regions showed increased incorporation into hnRNP particles (FIG. 4b). Long-range spaced binding across an exon, as seen in CD55 pre-mRNA (FIG. 2b), might silence splicing by incorporating the exon into the hnRNP particle. A related hypothesis proposed that binding of PTB via its four RRM domains to sites flanking an exon silences splicing by looping out the exon^{14,27,28}. In addition, we found that hnRNP particles enhance splicing when binding within the intron preceding the alternative exon (FIG. 4c). Thus, by incorporating long regions of RNA, hnRNP particles can play a dual role in splicing regulation. Importantly, the outcome of this regulation depends on the positioning of hnRNP particles on pre-mRNAs.

The RNA map of hnRNP C regulation described that silenced exons are flanked by precisely spaced cross-link nucleotides. In order to assess whether hnRNP C binding could predict silenced exons, we used the iCLIP data to search the transcriptome for exons that are flanked by hnRNP C cross-link nucleotides with a defined spacing of 160-170 nucleotides (FIG. 5a). We then chose nine alternatively spliced exons that had not shown hnRNP C-dependent regulation in our microarray analyses, and quantified their splicing behavior using RT-PCR. Strikingly, five of these (56%) showed significantly increased inclusion in hnRNP C knockdown cells (p value<0.05 by Student's t-test), while the others remained unchanged (FIG. 5b, Table 3). Thus, the hnRNP C binding patterns identified by the iCLIP data could predict exon silencing, further substantiating our model of position-dependent splicing regulation by hnRNP particles.

The broad distribution of hnRNP C cross-link sites over complete transcripts (FIG. 2c) suggested that the hnRNP C activity is not restricted to regulation of alternative splicing. Therefore, we analyzed hnRNP particle formation on constitutive exons and flanking intronic regions to find a similar coverage on exons and introns, as predicted by previous studies⁵. However, we found a decreased coverage at the splice sites, agreeing with the hypothesis that hnRNP particles need to be excluded from regions required for splicing⁷ (FIG. 4c). These results suggest that hnRNP particles maintain splicing fidelity by incorporating introns and exons, while leaving the splice sites free to interact with the splicing machinery. Global profiling of protein-RNA interactions has been successful in elucidating principles of post-transcriptional regulation. Over the past years, CLIP was proven as a powerful method to determine protein-RNA interactions in vivo on a global scale⁹⁻¹². However, the resolution of this method is limited due to the inability to directly identify the cross-linked nucleotides. Moreover, CLIP suffers from the inherent problem that most cDNAs truncate at the cross-link site and are thus lost during the amplification process. Here, we developed iCLIP, which overcomes these obstacles and identifies the positions of cross-link sites at nucleotide resolution. iCLIP also introduces a random barcode to mark individual cDNA molecules, thereby solving an inherent problem of all current high-throughput sequencing methods that suffer from PCR artefacts. Therefore, exploiting the random barcode strongly improves the quality of quantitative information. In order to identify clustered cross-link nucleotides, we applied a statistical algorithm to filter for enriched hnRNP C binding. Comparison of the clustered cross-link nucleotides with the complete dataset showed that both datasets generate similar results, suggesting that real binding sites constitute a major proportion of both. This observation underlines the high quality of iCLIP data, achieved by high stringency of purification and library preparation. Thus, iCLIP allows the transcriptome-wide analysis of protein-RNA interactions at individual nucleotide resolution.

We used iCLIP to show that hnRNP C binds to uridine tracts in nascent transcripts with a defined spacing of 165 and 300 nucleotides. These data agree with past findings that the hnRNP C tetramer binds in repetitive units of approximately 150-300 nucleotides^{6,23,24}. Whereas some studies suggested that this binding occurs in a sequence-independent manner^{6,23,24}, other studies proposed that the sequence-specific RRM domains critically contribute to high-affinity RNA binding of the hnRNP C tetramer¹⁷⁻¹⁹. iCLIP data agree with the latter model that hnRNP C is positioned on pre-mRNAs via sequence-specific binding of its RRM domains (FIG. 6). In addition, the precise spacing between the hnRNP C cross-link sites suggests that in accordance with the former model the basic leucine zipper-like RNA-binding motif (bZLM) domains guide the intervening RNA along the axis of the hnRNP C tetramer via sequence-independent electrostatic interactions^{22,29}. Thus, by measuring the spacing between distant binding sites, iCLIP can yield structural insights into ribonucleoprotein complexes.

Even though hnRNP particles were found to form on nuclear RNAs more than 30 years ago, their function in pre-mRNA processing remained unresolved⁴⁻⁸. Here, we present nucleotide-resolution mapping of in vivo hnRNP C cross-link sites, which reveals a role of hnRNP particles in splicing regulation. Importantly, we found that binding of hnRNP particles is guided by the pre-mRNA sequence to determine the splicing outcome in a position-dependent manner. In particular, alternative exons are silenced by incorporation into the hnRNP particles, whereas binding to the preceding intron enhances inclusion of alternative exons. Early studies had hypothesized that hnRNP particles might function to organize long introns for efficient splicing³⁰. This was based on the observation that long pre-mRNAs are highly compacted in hnRNP particles. In accordance with this hypothesis, we propose that hnRNP particles might act as ‘RNA nucleosomes’ that bind long regions of pre-mRNA, but maintain the correct splice sites accessible to the splicing machinery. The ability of iCLIP to study protein-RNA interactions with high resolution and in a quantitative manner holds promise for future studies of the structure and function of ribonucleoprotein complexes.

### REFERENCES

1. Nilsen, T. W. & Graveley, B. R. Expansion of the eukaryotic proteome by alternative splicing. Nature 463, 457-463.
2. Wahl, M. C., Will, C. L. & Lührmann, R. The spliceosome: design principles of a dynamic RNP machine. Cell 136, 701-718 (2009).
3. Chen, M. & Manley, J. L. Mechanisms of alternative splicing regulation: insights from molecular and genomics approaches. Nat Rev Mol Cell Biol 10, 741-754 (2009).
4. Beyer, A. L., Christensen, M. E., Walker, B. W. & LeStourgeon, W. M. Identification and characterization of the packaging proteins of core 40S hnRNP particles. Cell 11, 127-138 (1977).
5. Steitz, J. A. & Kamen, R. Arrangement of 30S heterogeneous nuclear ribonucleoprotein on polyoma virus late nuclear transcripts. Mol Cell Biol 1, 21-34 (1981).
6. Huang, M. et al. The C-protein tetramer binds 230 to 240 nucleotides of pre-mRNA and nucleates the assembly of 40S heterogeneous nuclear ribonucleoprotein particles. Mol Cell Biol 14, 518-533 (1994).
7. Reed, R. Mechanisms of fidelity in pre-mRNA splicing. Curr Opin Cell Biol 12, 340-345 (2000).
8. Amero, S. A. et al. Independent deposition of heterogeneous nuclear ribonucleoproteins and small nuclear ribonucleoprotein particles at sites of transcription. Proc Natl Acad Sci USA 89, 8409-8413 (1992).
9. Ule, J. et al. CLIP identifies Nova-regulated RNA networks in the brain. Science 302, 1212-1215 (2003).
10. Ule, J., Jensen, K., Mele, A. & Darnell, R. B. CLIP: A method for identifying protein-RNA interaction sites in living cells. Methods 37, 376-386 (2005).
11. Licatalosi, D. D. et al. HITS-CLIP yields genome-wide insights into brain alternative RNA processing. Nature 456, 464-469 (2008).
12. Yeo, G. W. et al. An RNA code for the FOX2 splicing regulator revealed by mapping RNA-protein interactions in stem cells. Nat Struct Mol Biol 16, 130-137 (2009).
13. Urlaub, H., Hartmuth, K. & Lührmann, R. A two-tracked approach to analyze RNA-protein crosslinking sites in native, nonlabeled small nuclear ribonucleoprotein particles. Methods 26, 170-181 (2002).
14. Xue, Y. et al. Genome-wide analysis of PTB-RNA interactions reveals a strategy used by the general splicing repressor to modulate exon inclusion or skipping. Mol Cell 36, 996-1006 (2009).
15. Kim, J. H. et al. Heterogeneous nuclear ribonucleoprotein C modulates translation of c-myc mRNA in a cell cycle phase-dependent manner. Mol Cell Biol 23, 708-720 (2003).
16. Zaidi, S. H. & Malter, J. S, Nucleolin and heterogeneous nuclear ribonucleoprotein C proteins specifically interact with the 3′-untranslated region of amyloid protein precursor mRNA. J Biol Chem 270, 17292-17298 (1995).
17. Gorlach, M., Wittekind, M., Beckman, R. A., Mueller, L. & Dreyfuss, G. Interaction of the RNA-binding domain of the hnRNP C proteins with RNA. EMBO J. 11, 3289-3295 (1992).
18. Gorlach, M., Burd, C. G. & Dreyfuss, G. The determinants of RNA-binding specificity of the heterogeneous nuclear ribonucleoprotein C proteins. J Biol Chem 269, 23074-23078 (1994).
19. Wan, L., Kim, J. K., Pollard, V. W. & Dreyfuss, G. Mutational definition of RNA-binding and protein-protein interaction domains of heterogeneous nuclear RNP C1. J Biol Chem 276, 7681-7688 (2001).
20. Hockensmith, J. W., Kubasek, W. L., Vorachek, W. R. & von Hippel, P. H. Laser cross-linking of nucleic acids to proteins. Methodology and first applications to the phage T4 DNA replication system. J Biol Chem 261, 3512-3518 (1986).
21. Hockensmith, J. W., Kubasek, W. L., Vorachek, W. R. & von Hippel, P. H. Laser cross-linking of proteins to nucleic acids. I. Examining physical parameters of protein-nucleic acid complexes. J Biol Chem 268, 15712-15720 (1993).
22. Whitson, S. R., LeStourgeon, W. M. & Krezel, A. M. Solution structure of the symmetric coiled coil tetramer formed by the oligomerization domain of hnRNP C: implications for biological function. J Mol Biol 350, 319-337 (2005).
23. Barnett, S. F., Friedman, D. L. & LeStourgeon, W. M. The C proteins of HeLa 40S nuclear ribonucleoprotein particles exist as anisotropic tetramers of (C1)3 C2. Mol Cell Biol 9, 492-498 (1989).
24. McAfee, J. G., Soltaninassab, S. R., Lindsay, M. E. & LeStourgeon, W. M. Proteins C1 and C2 of heterogeneous nuclear ribonucleoprotein complexes bind RNA in a highly cooperative fashion: support for their contiguous deposition on pre-mRNA during transcription. Biochemistry 35, 1212-1222 (1996).
25. Ule, J. et al. An RNA map predicting Nova-dependent splicing regulation. Nature 444, 580-586 (2006).
26. Singh, R., Valcarcel, J. & Green, M. R. Distinct binding specificities and functions of higher eukaryotic polypyrimidine tract-binding proteins. Science 268, 1173-1176 (1995).
27. Gooding, C., Roberts, G. C., Moreau, G., Nadal-Ginard, B. & Smith, C. W. Smooth muscle-specific switching of alpha-tropomyosin mutually exclusive exon selection by specific inhibition of the strong default exon. EMBO J. 13, 3861-3872 (1994).
28. Oberstrass, F. C. et al. Structure of PTB bound to RNA: specific binding and implications for splicing regulation. Science 309, 2054-2057 (2005).
29. McAfee, J. G., Shahied-Milam, L., Soltaninassab, S. R. & LeStourgeon, W. M. A major determinant of hnRNP C protein binding to RNA is a novel bZIP-like RNA binding domain. RNA 2, 1139-1152 (1996).
30. Choi, Y. D., Grabowski, P. J., Sharp, P. A. & Dreyfuss, G. Heterogeneous nuclear ribonucleoproteins: role in RNA splicing. Science 231, 1534-1539 (1986).
31. Langmead, B., Trapnell, C., Pop, M. & Salzberg, S. L. Ultrafast and memory-efficient alignment of short DNA sequences to the human genome. Genome Biol 10, R25 (2009).
32. Crooks, G. E., Hon, G., Chandonia, J. M. & Brenner, S. E. WebLogo: a sequence logo generator. Genome Res 14, 1188-1190 (2004).
33. Hubbard, T. J. et al. Ensembl 2009. Nucleic Acids Res 37, D690-697 (2009).
34. Ule, J. et al. Nova regulates brain-specific splicing to shape the synapse. Nat Genet. 37, 844-852 (2005).
35. Langmead, B., Trapnell, C., Pop, M. & Salzberg, S. L. Ultrafast and memory efficient alignment of short DNA sequences to the human genome. Genome Biol 10, R25 (2009).
36. Yeo, G. W. et al. An RNA code for the FOX2 splicing regulator revealed by mapping RNA-protein interactions in stem cells. Nat Struct Mol Biol 16, 130-137 (2009).

All publications mentioned in the above specification are herein incorporated by reference. Various modifications and variations of the described methods and system of the invention will be apparent to those skilled in the art without departing from the scope and spirit of the invention. Although the invention has been described hi connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

TABLE 1
Genomic mapping of iCLIP sequence reads.

| | | | |
|---|---|---|---|
| Replicate 1 | Replicate 2 | Replicate 3 | Total |

hnRNP C iCLIP experiments:

| | | | | |
|---|---|---|---|---|
| Initial sequence reads^{a} | 6544506 | 6544506 | 6544506 | 6544506 |
| After experiment separation | 2610554 | 2292169 | 1376258 | 6278981 |
| | | | | (96%)^{b} |
| After mapping to the human genome | 1595604 | 1624238 | 942970 | 4162812 |
| | | | | (66%) |
| After random barcode evaluation | 309489 | 216295 | 115566 | 641350 |
| | (19%) | (13%) | (12%) | (15%) |
| Cross-link nucleotides | 302692 | 212098 | 113920 | 614740^{c} |

No-antibody iCLIP controls:

| | | | | |
|---|---|---|---|---|
| Initial sequence reads | 5782612 | 12597621 | 12597621 | 18380233 |
| After experiment separation | 91310 | 122957 | 71044 | 285311 |
| | | | | (2%) |
| After mapping to the human genome | 6589 | 11055 | 15244 | 32888 |
| | | | | (11%) |
| After random barcode evaluation | 386 | 551 | 843 | 1780 |
| | (6%) | (5%) | (6%) | (5%) |
| Cross-link nucleotides | 384 | 520 | 803 | 1707 |

^{a}Number of sequence reads from Illumina GA2 before data analyses.
^{b}Numbers in brackets indicate fraction relative to entry above.
^{c}The total number of cross-link nucleotides is smaller than the sum of replicates 1-3. since reproduced positions were counted only once.

TABLE 2
Quantification of alternative mRNA isoforms using mRNA

| | | | | | | | | | | |
|---|---|---|---|---|---|---|---|---|---|---|
| | | | | | | % | | | | Product |
| Gene | | | | | | Microrray | % PCR | | | sizes in |
| symbol | Gene description | Exon | Spliced region | Exon coordinates | Strand | change | change | Forward primer | Reverse primer | bp (in/ex) |

Exons silenced by hnRNP C

| | | | | | | | | | | |
|---|---|---|---|---|---|---|---|---|---|---|
| A1590482 | n.a. | E2 | chr6:166282734- | chr6: 166258138- | − | −13.8 | no | AACTCGAAATGAAGCGGAAA | GCCTCCCTGTGAATTCTCTC; | 124/87 |
| | | | 166283392 | 166321007 | | | change | | TGGCTATTTTTGTTGATGATAGGA | |
| C20orf199 | Uncharacterized | E7 | chr20: 47330430- | chr20: 47329153- | + | −19.9 | −11.7 | TTGGAAGAGGGAGTCACCAC | TCCAGAGGGCTCCTCTCATA | 257/109 |
| | protein C20orf199 | | 47330514 | 47338969 | | | | | | |
| C6orf48 | Protein G8 | E13 | chr6: 31912181- | chr6: 31910957- | + | −57.5 | −58.6 | GTTCATCGCCGTGTTATCCT | GGGGGAGATTCCAAACCTTA | 214/120 |
| | | | 31912273 | 31912992 | | | | | | |
| CD55 | Complement decay- | E15 | chr1: 205580360- | chr1: 205579386- | + | −61.0 | −60.6 | CCAGGACAACCAAGCATTTT | GGAATCATCTTAAGTGTCCATCAA; | 407/114 |
| | accelerating factor | | 205580476 | 205599514 | | | | | CAAGCAAACCTGTCAACGTG | |
| | Precursor | | | | | | | | | |
| CEP57 | Centrosomal protein | E3 | chr11: 95168328- | chr11: 95163555- | + | −13.7 | −11.2 | CGGCTTCTGGTTCTCACTTG | GATGAAGAATGCCGAACCAT | 123/79 |
| | of 57 kDa | | 95168371 | 95172044 | | | | | | |
| CPSF1 | Cleavage and poly- | E5 | chr8: 145599070- | chr8: 145597675- | − | −36.0 | −27.2 | CTACGTGTACCGCCTCAACC | CGTTGCCAAAGAAGGAGAAG | 374/119 |
| | adenylation | | 145599193 | 145605207 | | | | | | |
| | specificity factor | | | | | | | | | |
| | subunit 1 | | | | | | | | | |
| DNMT1 | DNA (cytosine-5)- | E5 | chr19: 10151863- | chr19: 10149043- | − | −31.0 | −36.8 | GAAGCCCGTAGAGTGGGAAT | GCCTGGTGCTTTTCCTTGTA | 193/145 |
| | methyltransferase 1 | | 10151910 | 10152026 | | | | | | |
| EIF4A2 | Eukaryotic initiation | E6 | chr3: 187985179- | chr3: 187985179- | + | −12.7 | −14.6 | CCTTCCGCTATTCAGCAGAG | CAACTGTTGCAGGATGGAAA | 385/120 |
| | factor 4A-II | | 187985445 | 187985445 | | | | | | |
| GLS | Glutaminase kidney, | E17 | chr2: 191505688- | chr2: 191504609- | + | −31.5 | −19.1 | CCTCGAAGAGAAGGTGGTGA | CCTCATTTGACTCAGGTGACA; | 124/86 |
| | isoform mitochondrial | | 191508062 | 191526536 | | | | | CGAAGTGCAGACACATCTCC | |
| | Precursor | | | | | | | | | |
| NTNG1 | Netrin-G1 Precursor | E19 | chr1: 107774895- | chr1: 107762790- | + | −50.4 | no | CCCAAAGGCACTGCAAATAC; | AGCTCGTTGTCGCAGACATT | 188/71 |
| | | | 107775062 | 107824756 | | | change | GCACAACTGGACGATGAGAA | | |
| PCBP2 | Poly(rC)-binding | E14 | chr12: 52144811- | chr12: 52142618- | + | −22.4 | −13.4 | GTCATCTTTGCAGGTGGTCA | GCTTGGTCAAATCTGGCTGT | 166/73 |
| | protein 2 | | 52144903 | 52145983 | | | | | | |
| RBX1 | RING-box protein 1 | E4 | chr22: 39681237- | chr22: 39679583- | + | −40.7 | −13.0 | TGCAGGAACCACATTATGGA | CGAGAGATGCAGTGGAAGTG | 285/125 |
| | | | 39681395 | 39689997 | | | | | | |
| RCC1 | Regulator of chromo- | E6 | chr1: 28708001- | chr1: 28708001- | + | −56.1 | −40.6 | GATCTGCACTTCGCATTTTG | CCCTGGGATCTCTGATCTCTC; | 129/80 |
| | some condensation | | 28708004 | 28715824 | | | | | AAAAATCAGTTTACCTACTCT | |
| | | | | | | | | | CCCTTC | |
| SPIN1 | Spindin-1 | E6 | chr9: 90221380- | chr9: 90193274- | + | −32.8 | −8.5 | CCGTGGGCCTGTGGACTG | TCTGGTTAATCCACCATCCAA | 495/105 |
| | | | 90221501 | 90223513 | | | | | | |
| TMEM165 | Transmembrane | E3 | chr4: 55964161- | chr4: 55957321- | + | −30.4 | −16.9 | TAGCCACCGGAACAAAGAAC | GAACTGGAGCTGCTGGTGA | 222/119 |
| | protein 165 | | 55964262 | 55972538 | | | | | | |
| TXRND1 | Thioredoxin reductase | E12 | chr12: | chr12: | + | −23.5 | −14.9 | TTTTCTTCACTCCGGATTT | TCAGGGCCGTTCATTTTTAG | 358/136 |
| | 1, cytoplasmic | | 103208902- | 103205016- | | | | | | |
| | | | 103209012 | 103229198 | | | | | | |
| UBAP1 | Ubiquitin-associated | E5 | chr9: 34193380- | chr9: 34169239- | + | −24.0 | −33.8 | CACCTTTCGGCTTCTGAGAC | CATGAAAATCTGCACCCAACT | 205/83 |
| | protein 1 | | 34193533 | 34210906 | | | | | | |
| ZNF145 | Zinc finger protein | E4 | chr19: 41400864- | chr19: 41397938- | + | −29.0 | −11.6 | CCGAGTGACATTTTGGTCT | TTCTTGCTTCAACAGAGGATCA | 132/58 |
| | OZF | | 41400937 | 41411490 | | | | | | |

Exons enhanced by hnRNP C

| | | | | | | | | | | |
|---|---|---|---|---|---|---|---|---|---|---|
| EIF4G2 | Eukaryotic | E20 | chr11: 10779764- | chr11: 10779210- | − | 25.1 | 22.9 | ATCGCAGTTTGGAGAGATGG | TATCTGGGGCTGAAGCTTTG | 226/112 |
| | translation | | 10779897 | 10780172 | | | | | | |
| | initiation | | | | | | | | | |
| | factor 4 gamma 2 | | | | | | | | | |
| FNBP4 | Formin-binding | E19 | chr: 11: 47703866- | chr11: 47702907- | − | 20.3 | 12.9 | TTGCCAAACAGACCTTGAAA | GGAGGGTCCAGAATGGAGTA | 250/150 |
| | protein 4 | | 47703964 | 47709502 | | | | | | |
| MFF | Mitochondrial | E12 | chr2: 227920186- | chr2: 227913340- | + | 24.1 | 14.3 | GAAGGAAATCCGAGCAGTTGG | TGACGTTCCTTCAATGGTTG | 357/138 |
| | fission factor | | 227920344 | 227928637 | | | | | | |
| NUP98 | Nuclear pore complex | E13 | chr11: 3722316- | chr11: 3713131- | − | 20.9 | 28.0 | TAAACCAGCACCTGGGACTC | ATTGATGTGCTGCTGGAGAA | 253/112 |
| | protein Nup96-Nup96 | | 3722455 | 3731122 | | | | | | |
| | Precursor | | | | | | | | | |
| PUM2 | Pumilio homolog 2 | E14 | chr2: 20341825- | chr2: 20326702- | − | 24.5 | 15.5 | GGGTGCTGCTATAGGTCAG | CTCCAGGTGCTGCAGAGATA | 330/93 |
| | | | 20342061 | 20346189 | | | | | | |
| SLMAP | sarcolemmal membrane- | E14 | chr3: 57826009- | chr3: 57825483- | + | 45.6 | 22.6 | GGAGCTCCAGGCAAAAATAG | TTGGTTAGATGCCCTTCGAC | 270/168 |
| | associated protein | | 57826059 | 57832403 | | | | | | |
| SNRPN | Small nuclear | E43 | chr15: 22798965- | chr15: 22778677- | + | 18.6 | 12.3 | GTGATGTCCAGGAAGGAGGA | TGATTCCATTTGCAGGTCAG | 229/107 |
| | ribonucleoprotein- | | 22799128 | 22815267 | | | | | | |
| | associated protein N | | | | | | | | | |
| TRPS1 | Zinc finger | E3 | chr8: 116705004- | chr8: 116701463- | − | 29.4 | 21.0 | CGAGGGTGTTCTTGACGATT | CCTTCACTTGCAACGTTTCTC | 236/78 |
| | transcription factor | | 116705160 | 116749946 | | | | | | |
| | Trps1 | | | | | | | | | |

TABLE 3
Quantification of predicted splicing changes using RT-PCR

| | | | | | | | | | | |
|---|---|---|---|---|---|---|---|---|---|---|
| | | | | | | | | | | Product |
| | | | Alternative | Coordinates | | | | | | sizes |
| Gene | Gene | | exon | of skipped | | % PCR | p | Forward | Reverse | in by |
| symbol | description | Exon | coordinates | region | Strand | change | value | primer | primer | (in/ex) |

Exons silenced by hnRNP C

| | | | | | | | | | | |
|---|---|---|---|---|---|---|---|---|---|---|
| C12orf23 | UPF0444 | E7 | chr12: | chr12: | + | −19.9 | 3.9 × | CCTTAATGATG | AAGATACCCC | 87/206 |
| | transmembrane | | 105885192- | 105885089- | | | 10⁶ | AACCACCAGAA | CAGTCACACG | |
| | protein | | 105885309 | 105889013 | | | | | | |
| | C12orf23 | | | | | | | | | |
| MTRF1 | Peptide chain | E3 | chr13: | chr13: | − | −23.5 | 1.7 × | TTCCGACCTCA | CCAAACACAC | 79/150 |
| | release | | 40734548- | 40734468- | | | 10² | GTAAAGAGAGC | AGGTGACGAT | |
| | factor 1, | | 40734617 | 40735621 | | | | | | |
| | mitochominal | | | | | | | | | |
| | Precursor | | | | | | | | | |
| PRKAA1 | 5-AMP- | E6 | chr5: | chr5: | − | −6.2 | 1.8 × | TGTCTCAGGAG | GACGCCGACT | 71/116 |
| | activated | | 40810788- | 40807723- | | | 10³ | GAGAGCTATT | TTCTTTTTCA | |
| | protein | | 40810831 | 40811269 | | | | TG | | |
| | kinase | | | | | | | | | |
| | catalytic | | | | | | | | | |
| | subunit | | | | | | | | | |
| | alpha-1 | | | | | | | | | |
| TBL1XR1 | F-box-like/ | E3 | chr3: | chr3: | − | −17.1 | 1.0 × | GTTGGAGGCCA | TGCAACTGAA | 70/188 |
| | WD repeat- | | 178361354- | 178299024- | | | 10³ | CCGTTTC | TATCCGGTCA | |
| | containing | | 178361470 | 178397603 | | | | | | |
| | protein | | | | | | | | | |
| | TBL1XR1 | | | | | | | | | |
| ZNF195 | Zinc finger | E7 | chr11: | chr11: | − | −18.5 | 1.0 × | AGCCCTGGAA | CTGGCAGAAG | 81/185 |
| | protein 195 | | 3347164- | 3340409- | | | 10³ | TGTGAAGAGA | GTCTTGGGTA | |
| | | | 3347308 | 3348781 | | | | | ACGCAGCAAT | |
| | | | | | | | | | CACACTTCTG | |

no change observed

| | | | | | | | | | | |
|---|---|---|---|---|---|---|---|---|---|---|
| BRD2 | Bromodomain- | E16 | chr6: | chr6: | + | 3.3 | 0.2 | TGGACCTTCT | CTGTAGGCAG | 74/179 |
| | containing | | 33054846- | 33054144- | | | | GGAGGAAGTG | GGCAGGTG | |
| | protein 2 | | 33054933 | 33055583 | | | | | | |
| CHD2 | Chromodomain- | E3 | chr15: | chr15: | + | −4.8 | 0.11 | GGTTTGGGCG | CAGAACCAAC | 86/142 |
| | helicase-DNA- | | 91227852- | 91227778- | | | | ACCAGGAG | AGCAACCAAA | |
| | binding | | 91228012 | 91229749 | | | | | TGAAACGTAGT | |
| | protein 2 | | | | | | | | CAGGGTTCCA | |
| FLNB | Filamin-B | E30 | chr3: | chr3: | + | 1.5 | 0.23 | TCCTAACAGCC | CAGGCCGTTC | 70/142 |
| | | | 58102626- | 58099297- | | | | CCTTCACTG | ATGTCACTC | |
| | | | 58102663 | 58103417 | | | | | | |
| IQWD1 | Nuclear | E16 | chr1: | chr1: | + | 3.1 | 0.14 | TCTGTTGAGGC | GTTCACCTGT | 85/145 |
| | receptor | | 166258851- | 166240656- | | | | ATCTGGACA | CCCTGGTTTG | |
| | interaction | | 166258909 | 166274233 | | | | | | |

1. A method for identifying an interaction between an RNA and an RNA binding protein in a biological sample, comprising the steps of:
a) contacting the biological sample with an agent that creates a covalent bond between the RNA and the RNA binding protein to form cross-linked RNA;
b) fragmenting said RNA;
c) ligating a first adapter to the fragmented RNA;
d) hybridising a reverse transcription primer to said first adapter and reverse transcribing said cross-linked RNA into cDNA;
e) circularising the transcribed cDNA;
f) linearising the circularised cDNA; and
g) determining the sequence of one or more of the cDNAs.

2. The method of claim 1, wherein the covalent bond between the RNA and the RNA binding protein is created by cross-linking.

3. The method according to claim 1, wherein the reverse transcription primer comprises a cleavable adapter.

4. The method according to claim 3, wherein the reverse transcription primer comprises two inversely orientated adapter regions separated by a cleavable adapter.

5. The method according to claim 3, wherein the cleavable adapter is cleavable by a restriction enzyme.

6. The method according to claim 3, wherein said cleavable adapter additionally comprises one or more nucleotides of known or unknown sequence as an experiment identifier and/or to identify amplification duplicates.

7. The method according to claim 6, wherein the one or more nucleotides of known or unknown sequence as an experiment identifier comprises at least two nucleotides.

8. The method according to claim 6, wherein the one or more nucleotides of known or unknown sequence to identify amplification duplicates comprise at least three nucleotides.

9. The method according to claim 1, wherein cDNA sequences that truncate at the same nucleotide in the genome and share the same one or more nucleotides of known or unknown sequence to identify amplification duplicates are eliminated from subsequent analysis.

10. The method according to claim 3, wherein the circularised cDNA is linearised at the cleavable adapter.

11. The method according to claim 1, wherein a primer complementary to at least a portion of the reverse transcription primer is hybridised thereto prior to linearisation.

12. The method according to claim 1, wherein the cDNA is amplified by hybridising one or more primers that are complementary in sequence to at least a portion of the cleaved adapter.

13. The method according to claim 1, wherein the nucleotide sequence of the amplified cDNA is determined up to the point that the cDNAs truncate at the crosslink site thereby providing individual nucleotide resolution of the crosslinking site.

14. The method according to claim 13, wherein the nucleotide sequence of 5 or more of the nucleotides of the amplified cDNA up to the point that the cDNAs truncate at the crosslink site is determined.

15. A method for preparing a cDNA library representative of one or more interactions between an RNA and an RNA binding protein, comprising the steps of:
a) contacting the biological sample with an agent that creates a covalent bond between the RNA and the RNA binding protein;
b) fragmenting said RNA;
c) ligating a first adapter to the fragmented RNA;
d) hybridising a reverse transcription primer to said first adapter and reverse transcribing said cross-linked RNA;
e) circularising the transcribed cDNA;
f) optionally linearising the circularised cDNA; and
g) optionally sub-cloning the linearised cDNA into a vector.

16. A method of mapping one or more interactions between an RNA and an RNA binding protein, comprising the steps of:
identifying an interaction between an RNA and an RNA binding protein in a biological sample according to the method of claim 1; and
b) determining the location of the interaction in the genome.

17. The method according to claim 16, wherein mapping of the interaction(s) is performed against the human genome to determine the position of crosslink nucleotides.

18. The method according to claim 16, wherein mapping of the interaction(s) is based on sequences that map to human nuclear chromosomes.

19. The method according to claim 16, wherein amplification duplicates are excluded.

20. The method according to claim 16, wherein the interaction(s) between RNA and an RNA binding protein are determined in replicate.


SEQUENCE LISTING (102 sequences)

SEQ ID NO: 1 - DNA, 22 bp, Artificial sequence
  Synthetic sequence Adapter
ugagaucggaagagcggttcag

SEQ ID NO: 2 - DNA, 34 bp, Artificial sequence
  Synthetic sequence Reverse transcription primer
agatcggaagagcgtcgtggatcctgaaccgctc

SEQ ID NO: 3 - DNA, 37 bp, Artificial sequence
  Synthetic sequence Reverse transcription primer
nnnagatcggaagagcgtcgtggatcctgaaccgctc

SEQ ID NO: 4 - DNA, 39 bp, Artificial sequence
  Synthetic sequence Reverse transcription primer
nnncaagatcggaagagcgtcgtggatcctgaaccgctc

SEQ ID NO: 5 - DNA, 32 bp, Artificial sequence
  Synthetic sequence Reverse transcription primer
agatcggaagagcgtcgtggatcctgaaccgc

SEQ ID NO: 6 - DNA, 35 bp, Artificial sequence
  Synthetic sequence Reverse transcription primer
nnnagatcggaagagcgtcgtggatcctgaaccgc

SEQ ID NO: 7 - DNA, 37 bp, Artificial sequence
  Synthetic sequence Reverse transcription primer
nnngaagatcggaagagcgtcgtggatcctgaaccgc

SEQ ID NO: 8 - DNA, 34 bp, Artificial sequence; identical to SEQ ID NO: 2
  Synthetic sequence Reverse transcription primer

SEQ ID NO: 9 - DNA, 37 bp, Artificial sequence; identical to SEQ ID NO: 3
  Synthetic sequence Reverse transcription primer

SEQ ID NO: 10 - DNA, 39 bp, Artificial sequence
  Synthetic sequence Reverse transcription primer
nnntgagatcggaagagcgtcgtggatcctgaaccgctc

SEQ ID NO: 11 - DNA, 27 bp, Artificial sequence
  Synthetic sequence Linearisation primer
gttcaggatccacgacgctcttcaaaa

SEQ ID NO: 12 - DNA, 61 bp, Artificial sequence
  Synthetic sequence Primer
caagcagaagacggcatacgagatcggtctcggcattcctgctgaaccgctcttccgatct

SEQ ID NO: 13 - DNA, 58 bp, Artificial sequence
  Synthetic sequence Primer
aatgatacggcgaccaccgagatctacactctttccctacacgacgctcttccgatct

SEQ ID NO: 14 - DNA, 20 bp, Artificial sequence
  Synthetic sequence Primer
aactcgaaatgaagcggaaa

SEQ ID NO: 15 - DNA, 20 bp, Artificial sequence
  Synthetic sequence Primer
gcctccctgtgaattctctc

SEQ ID NO: 16 - DNA, 24 bp, Artificial sequence
  Synthetic sequence Primer
tggctatttttgttgatgatagga

SEQ ID NO: 17 - DNA, 20 bp, Artificial sequence
  Synthetic sequence Primer
ttggaagagggagtcaccac

SEQ ID NO: 18 - DNA, 20 bp, Artificial sequence
  Synthetic sequence Primer
tccagagggctcctctcata

SEQ ID NO: 19 - DNA, 20 bp, Artificial sequence
  Synthetic sequence Primer
gttcatcgccgtgttatcct

SEQ ID NO: 20 - DNA, 20 bp, Artificial sequence
  Synthetic sequence Primer
gggggagattccaaacctta

SEQ ID NO: 21 - DNA, 20 bp, Artificial sequence
  Synthetic sequence Primer
ccaggacaaccaagcatttt

SEQ ID NO: 22 - DNA, 24 bp, Artificial sequence
  Synthetic sequence Primer
ggaatcatcttaagtgtccatcaa

SEQ ID NO: 23 - DNA, 20 bp, Artificial sequence
  Synthetic sequence Primer
caagcaaacctgtcaacgtg

SEQ ID NO: 24 - DNA, 20 bp, Artificial sequence
  Synthetic sequence Primer
cggcttctggttctcacttg

SEQ ID NO: 25 - DNA, 20 bp, Artificial sequence
  Synthetic sequence Primer
gatgaagaatgccgaaccat

SEQ ID NO: 26 - DNA, 20 bp, Artificial sequence
  Synthetic sequence Primer
ctacgtgtaccgcctcaacc

SEQ ID NO: 27 - DNA, 20 bp, Artificial sequence
  Synthetic sequence Primer
cgttgccaaagaaggagaag

SEQ ID NO: 28 - DNA, 20 bp, Artificial sequence
  Synthetic sequence Primer
gaagcccgtagagtgggaat

SEQ ID NO: 29 - DNA, 20 bp, Artificial sequence
  Synthetic sequence Primer
gcctggtgcttttccttgta

SEQ ID NO: 30 - DNA, 20 bp, Artificial sequence
  Synthetic sequence Primer
ccttccgctattcagcagag

SEQ ID NO: 31 - DNA, 20 bp, Artificial sequence
  Synthetic sequence Primer
caactgttgcaggatggaaa

SEQ ID NO: 32 - DNA, 20 bp, Artificial sequence
  Synthetic sequence Primer
cctcgaagagaaggtggtga

SEQ ID NO: 33 - DNA, 21 bp, Artificial sequence
  Synthetic sequence Primer
cctcatttgactcaggtgaca

SEQ ID NO: 34 - DNA, 20 bp, Artificial sequence
  Synthetic sequence Primer
cgaagtgcagacacatctcc

SEQ ID NO: 35 - DNA, 20 bp, Artificial sequence
  Synthetic sequence Primer
cccaaaggcactgcaaatac

SEQ ID NO: 36 - DNA, 20 bp, Artificial sequence
  Synthetic sequence Primer
gcacaactggacgatgagaa

SEQ ID NO: 37 - DNA, 20 bp, Artificial sequence
  Synthetic sequence Primer
agctcgttgtcgcagacatt

SEQ ID NO: 38 - DNA, 20 bp, Artificial sequence
  Synthetic sequence Primer
gtcatctttgcaggtggtca

SEQ ID NO: 39 - DNA, 20 bp, Artificial sequence
  Synthetic sequence Primer
gcttggtcaaatctggctgt

SEQ ID NO: 40 - DNA, 20 bp, Artificial sequence
  Synthetic sequence Primer
tgcaggaaccacattatgga

SEQ ID NO: 41 - DNA, 20 bp, Artificial sequence
  Synthetic sequence Primer
cgagagatgcagtggaagtg

SEQ ID NO: 42 - DNA, 20 bp, Artificial sequence
  Synthetic sequence Primer
gatctgcacttcgcattttg

SEQ ID NO: 43 - DNA, 21 bp, Artificial sequence
  Synthetic sequence Primer
ccctgggatctctgatctctc

SEQ ID NO: 44 - DNA, 27 bp, Artificial sequence
  Synthetic sequence Primer
aaaaatcagtttacctactctcccttc

SEQ ID NO: 45 - DNA, 18 bp, Artificial sequence
  Synthetic sequence Primer
ccgtgggcctgtggactg

SEQ ID NO: 46 - DNA, 21 bp, Artificial sequence
  Synthetic sequence Primer
tctggttaatccaccatccaa

SEQ ID NO: 47 - DNA, 20 bp, Artificial sequence
  Synthetic sequence Primer
tagccaccggaacaaagaac

SEQ ID NO: 48 - DNA, 20 bp, Artificial sequence
  Synthetic sequence Primer
gaactggagctgctggtgta

SEQ ID NO: 49 - DNA, 20 bp, Artificial sequence
  Synthetic sequence Primer
ttttcttcactccggcattt

SEQ ID NO: 50 - DNA, 20 bp, Artificial sequence
  Synthetic sequence Primer
tcagggccgttcatttttag

SEQ ID NO: 51 - DNA, 20 bp, Artificial sequence
  Synthetic sequence Primer
cacctttcggcttctgagac

SEQ ID NO: 52 - DNA, 21 bp, Artificial sequence
  Synthetic sequence Primer
catgaaaatctgcacccaact

SEQ ID NO: 53 - DNA, 20 bp, Artificial sequence
  Synthetic sequence Primer
ccgagtggacattttggtct

SEQ ID NO: 54 - DNA, 22 bp, Artificial sequence
  Synthetic sequence Primer
ttcttgcttcaacagaggatca

SEQ ID NO: 55 - DNA, 20 bp, Artificial sequence
  Synthetic sequence Primer
atcgcagtttggagagatgg

SEQ ID NO: 56 - DNA, 20 bp, Artificial sequence
  Synthetic sequence Primer
tatctggggctgaagctttg

SEQ ID NO: 57 - DNA, 20 bp, Artificial sequence
  Synthetic sequence Primer
ttgccaaacagaccttgaaa

SEQ ID NO: 58 - DNA, 20 bp, Artificial sequence
  Synthetic sequence Primer
ggagggtccagaatggagta

SEQ ID NO: 59 - DNA, 20 bp, Artificial sequence
  Synthetic sequence Primer
gaagaaatccgagcagttgg

SEQ ID NO: 60 - DNA, 20 bp, Artificial sequence
  Synthetic sequence Primer
tgacgttccttcaatggttg

SEQ ID NO: 61 - DNA, 20 bp, Artificial sequence
  Synthetic sequence Primer
taaaccagcacctgggactc

SEQ ID NO: 62 - DNA, 20 bp, Artificial sequence
  Synthetic sequence Primer
attgatgtgctgctggagaa

SEQ ID NO: 63 - DNA, 20 bp, Artificial sequence
  Synthetic sequence Primer
gggtgctgctataggctcag

SEQ ID NO: 64 - DNA, 20 bp, Artificial sequence
  Synthetic sequence Primer
ctccaggtgctgcagagata

SEQ ID NO: 65 - DNA, 20 bp, Artificial sequence
  Synthetic sequence Primer
ggagctccaggcaaaaatag

SEQ ID NO: 66 - DNA, 20 bp, Artificial sequence
  Synthetic sequence Primer
ttggttagatgcccttcgac

SEQ ID NO: 67 - DNA, 20 bp, Artificial sequence
  Synthetic sequence Primer
gtgatgtccaggaaggagga

SEQ ID NO: 68 - DNA, 20 bp, Artificial sequence
  Synthetic sequence Primer
tgattccatttgcaggtcag

SEQ ID NO: 69 - DNA, 20 bp, Artificial sequence
  Synthetic sequence Primer
cgagggtgttcttgacgatt

SEQ ID NO: 70 - DNA, 21 bp, Artificial sequence
  Synthetic sequence Primer
ccttcacttgcaacgtttctc

SEQ ID NO: 71 - DNA, 22 bp, Artificial sequence
  Synthetic sequence Primer
ccttaatgatgaaccaccagaa

SEQ ID NO: 72 - DNA, 20 bp, Artificial sequence
  Synthetic sequence Primer
aagatacccccagtcacacg

SEQ ID NO: 73 - DNA, 22 bp, Artificial sequence
  Synthetic sequence Primer
ttccgacctcagtaaagagagc

SEQ ID NO: 74 - DNA, 20 bp, Artificial sequence
  Synthetic sequence Primer
ccaaacacacaggtgacgat

SEQ ID NO: 75 - DNA, 23 bp, Artificial sequence
  Synthetic sequence Primer
tgtctcaggaggagagctatttg

SEQ ID NO: 76 - DNA, 20 bp, Artificial sequence
  Synthetic sequence Primer
gacgccgactttctttttca

SEQ ID NO: 77 - DNA, 18 bp, Artificial sequence
  Synthetic sequence Primer
gttggaggccaccgtttc

SEQ ID NO: 78 - DNA, 20 bp, Artificial sequence
  Synthetic sequence Primer
tgcaactgaatatccggtca

SEQ ID NO: 79 - DNA, 20 bp, Artificial sequence
  Synthetic sequence Primer
agccctggaatgtgaagaga

SEQ ID NO: 80 - DNA, 20 bp, Artificial sequence
  Synthetic sequence Primer
ctggcagaaggtcttgggta

SEQ ID NO: 81 - DNA, 20 bp, Artificial sequence
  Synthetic sequence Primer
acgcagcaatcacacttctg

SEQ ID NO: 82 - DNA, 20 bp, Artificial sequence
  Synthetic sequence Primer
tggaccttctggaggaagtg

SEQ ID NO: 83 - DNA, 18 bp, Artificial sequence
  Synthetic sequence Primer
ctgtaggcagggcaggtg

SEQ ID NO: 84 - DNA, 18 bp, Artificial sequence
  Synthetic sequence Primer
ggtttgggcgaccaggag

SEQ ID NO: 85 - DNA, 20 bp, Artificial sequence
  Synthetic sequence Primer
cagaaccaacagcaaccaaa

SEQ ID NO: 86 - DNA, 21 bp, Artificial sequence
  Synthetic sequence Primer
tgaaacgtagtcagggttcca

SEQ ID NO: 87 - DNA, 20 bp, Artificial sequence
  Synthetic sequence Primer
tcctaacagccccttcactg

SEQ ID NO: 88 - DNA, 19 bp, Artificial sequence
  Synthetic sequence Primer
caggccgttcatgtcactc

SEQ ID NO: 89 - DNA, 20 bp, Artificial sequence
  Synthetic sequence Primer
tctgttgaggcatctggaca

SEQ ID NO: 90 - DNA, 20 bp, Artificial sequence
  Synthetic sequence Primer
gttcacctgtccctggtttg

SEQ ID NO: 91 - DNA, 66 bp, Homo sapiens
atttgttttctttcttttcttttttttatttttattttttttttgagacaggttctcgtcctgtca

SEQ ID NO: 92 - DNA, 16 bp, Homo sapiens
attttatttttttttg

SEQ ID NO: 93 - DNA, 16 bp, Homo sapiens
attttttgtattttta

SEQ ID NO: 94 - DNA, 21 bp, Homo sapiens
caaaaaaaaaaaaaaaaaaag

SEQ ID NO: 95 - DNA, 24 bp, Homo sapiens
taaaaaaagattagtttaaaaaat

SEQ ID NO: 96 - DNA, 14 bp, Homo sapiens
gaaaaagtatgggc

SEQ ID NO: 97 - DNA, 13 bp, Homo sapiens
gaaaccaaaaaat

SEQ ID NO: 98 - DNA, 14 bp, Homo sapiens
taaaaatacaaaag

SEQ ID NO: 99 - DNA, 16 bp, Homo sapiens
caaaaaaaaaaaaaat

SEQ ID NO: 100 - DNA, 22 bp, Homo sapiens
taaaagttagaaaaagataaac

SEQ ID NO: 101 - DNA, 12 bp, Homo sapiens
gataaagaaaat

SEQ ID NO: 102 - DNA, 52 bp, Homo sapiens
cagctgcttagacgctggatttttttcgggtagtggaaaaccaggtaagcac